SEQUENCE LISTING (20 sequences)

SEQ ID NO: 1 - protein, 109 aa, Homo sapiens
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 2 - protein, 375 aa, Human
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 3 - DNA, 747 bp, Homo sapiens
caggtgcagctggtgcaatctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagacgagaactgggggttcgacccctggggccagggaaccctggtcaccgtctcgagtggaggcggcggttcaggcggaggtggctctggcggtggcggaagtgcactttcctatgagctgactcagccaccctcagtgtccgtgtctccaggacagacagccaccattacctgctctggacatgcactgggggacaaatttgtttcctggtatcagcagggatcaggccagtcccctgtattggtcatctatgacgatacccagcggccctcagggatccctgggcgattctctggctccaactctgggaacacagccactctgaccatcagcgggacccaggctatggatgaggctgactatttttgtcaggcgtgggacagcagcttcgtattcggcggagggaccaaggtcaccgtcctaggtgcggccgcacatcatcatcaccatcac

SEQ ID NO: 4 - protein, 249 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDENWGFDPWGQGTLVTVSSGGGGSGGGGSGGGGSALSYELTQPPSVSVSPGQTATITCSGHALGDKFVSWYQQGSGQSPVLVIYDDTQRPSGIPGRFSGSNSGNTATLTISGTQAMDEADYFCQAWDSSFVFGGGTKVTVLGAAAHHHHHH

SEQ ID NO: 5 - DNA, 351 bp, Homo sapiens
caggtgcagctggtgcaatctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagacgagaactgggggttcgacccctggggccagggaaccctggtcaccgtctcgagt

SEQ ID NO: 6 - protein, 117 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDENWGFDPWGQGTLVTVSS

SEQ ID NO: 7 - DNA, 315 bp, Homo sapiens
tcctatgagctgactcagccaccctcagtgtccgtgtctccaggacagacagccaccattacctgctctggacatgcactgggggacaaatttgtttcctggtatcagcagggatcaggccagtcccctgtattggtcatctatgacgatacccagcggccctcagggatccctgggcgattctctggctccaactctgggaacacagccactctgaccatcagcgggacccaggctatggatgaggctgactatttttgtcaggcgtgggacagcagcttcgtattcggcggagggaccaaggtcaccgtccta

SEQ ID NO: 8 - protein, 105 aa, Homo sapiens
SYELTQPPSVSVSPGQTATITCSGHALGDKFVSWYQQGSGQSPVLVIYDDTQRPSGIPGRFSGSNSGNTATLTISGTQAMDEADYFCQAWDSSFVFGGGTKVTVL

SEQ ID NO: 9 - DNA, 747 bp, Homo sapiens
caggtgcagctggtgcaatctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagacgagaactgggggttcgacccctggggccagggaaccctggtcaccgtctcgagtggaggcggcggttcaggcggaggtggctctggcggtggcggaagtgcactttcctatgagctgactcagccaccctcagtgtccgtgtctccaggacagacagccagcattacctgctctggacatgcactgggggacaaatttgtttcctggtatcagcagaagccaggccagtcccctgtattggtcatctatgacgatacccagcggccctcagggatccctgagcgattctctggctccaactctgggaacacagccactctgaccatcagcgggacccaggctatggatgaggctgactattactgtcaggcgtgggacagcagcttcgtattcggcggagggaccaaggtcaccgtcctaggtgcggccgcacatcaccatcaccatcac

SEQ ID NO: 10 - protein, 249 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDENWGFDPWGQGTLVTVSSGGGGSGGGGSGGGGSALSYELTQPPSVSVSPGQTASITCSGHALGDKFVSWYQQKPGQSPVLVIYDDTQRPSGIPERFSGSNSGNTATLTISGTQAMDEADYYCQAWDSSFVFGGGTKVTVLGAAAHHHHHH

SEQ ID NO: 11 - DNA, 351 bp, Homo sapiens; identical to SEQ ID NO: 5

SEQ ID NO: 12 - protein, 117 aa, Homo sapiens; identical to SEQ ID NO: 6

SEQ ID NO: 13 - DNA, 315 bp, Homo sapiens
tcctatgagctgactcagccaccctcagtgtccgtgtctccaggacagacagccagcattacctgctctggacatgcactgggggacaaatttgtttcctggtatcagcagaagccaggccagtcccctgtattggtcatctatgacgatacccagcggccctcagggatccctgagcgattctctggctccaactctgggaacacagccactctgaccatcagcgggacccaggctatggatgaggctgactattactgtcaggcgtgggacagcagcttcgtattcggcggagggaccaaggtcaccgtccta

SEQ ID NO: 14 - protein, 105 aa, Homo sapiens
SYELTQPPSVSVSPGQTASITCSGHALGDKFVSWYQQKPGQSPVLVIYDDTQRPSGIPERFSGSNSGNTATLTISGTQAMDEADYYCQAWDSSFVFGGGTKVTVL

SEQ ID NO: 15 - protein, 5 aa, Homo sapiens
SYYMH

SEQ ID NO: 16 - protein, 17 aa, Homo sapiens
IINPSGGSTSYAQKFQG

SEQ ID NO: 17 - protein, 8 aa, Homo sapiens
DENWGFDP

SEQ ID NO: 18 - protein, 11 aa, Homo sapiens
SGHALGDKFVS

SEQ ID NO: 19 - protein, 7 aa, Homo sapiens
DDTQRPS

SEQ ID NO: 20 - protein, 7 aa, Homo sapiens
QAWDSSF

(19) United States
(12) Patent Application Publication
(10) Pub. No.: US 2006/0240487 A1
(43) Pub. Date: Oct. 26, 2006

(54) Detection of GDF-8 modulating agents

(76) Inventors: John A. Nowak, Stratham, NH (US); John G. Cryan, Shrewsbury, MA (US); Kristin F. Murray, Townsend, MA (US); Joseph W. Rajewski III, South Boston, MA (US); Shujun Sun, Brentwood, NH (US); Neil M. Wolfman, Dover, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/387,643

(22) Filed: Mar. 23, 2006

Related U.S. Application Data
(60) Provisional application No. 60/664,400, filed on Mar. 23, 2005.

(51) Int. Cl. G01N 33/53

(52) U.S. Cl. 435/7.5

(57) ABSTRACT

Methods to detect GDF-8 modulating agents in animals, including humans, are provided herein, including methods to detect the presence of exogenous GDF-8 modulating agent such as a GDF-8 inhibitor in a biological sample. In particular, methods to assess the presence and/or quantity of a GDF-8 modulating agent in a biological sample are provided.

### RELATED APPLICATIONS

This application claims the benefit of U.S. Provisional Application No. 60/664,400, filed on Mar. 23, 2005, the contents of which are incorporated herein in their entirety by reference.

### BACKGROUND

Growth and differentiation factor-8 (GDF-8), also known as myostatin, is a secreted protein that is a negative regulator of skeletal muscle mass. Inhibitors of GDF-8 increase muscle growth, and are potentially beneficial in the treatment of a variety of conditions including sarcopenia, cachexia, and muscular dystrophy.

GDF-8 is a member of the transforming growth factor-beta (TGF-β) superfamily of structurally related growth factors. Members of this superfamily possess physiologically important growth-regulatory and morphogenetic properties (Kingsley et al., Genes Dev. 8:133-146 (1994); Hoodless et al., Curr. Topics Microbiol. Immunol. 228:235-272 (1998)). Similarly, they share a common structural organization including a short peptide signal for secretion and an amino-terminal portion separated from a bioactive carboxy-terminal portion by a highly conserved proteolytic cleavage site.

Human GDF-8 is synthesized as a 375 amino acid precursor protein that includes an amino-terminal propeptide portion and a carboxy-terminal mature portion. The propeptide is cleaved from mature GDF-8 at Arg-266. The mature GDF-8 protein is active as a disulfide linked homodimer. Following proteolytic processing, it is believed that two GDF-8 propeptides remain non-covalently complexed with the GDF-8 mature domain dimer, maintaining GDF-8 in a latent, inactive state (Lee et al., Proc. Natl. Acad. Sci. U.S.A. 98:9306-9311 (2001); Thies et al., Growth Factors 18:251-259 (2001)). Other proteins are also known to bind to mature GDF-8 and inhibit its biological activity. Such inhibitory proteins include follistatin and follistatin-related proteins, including GASP-1 (Gamer et al., Dev. Biol. 208:222-232 (1999)); U.S. Patent Pub. No. 2003-0180306-A1; U.S. Patent Pub. No. 2003-0162714-A1).

An alignment of deduced amino acid sequences from various species demonstrates that GDF-8 is highly conserved throughout evolution (McPherron et al., Proc. Nat. Acad. Sci. U.S.A. 94:12457-12461 (1997)). In fact, the sequences of human, mouse, rat, porcine, and chicken GDF-8 are 100% identical in the carboxy-terminal region, while in baboon, bovine, and ovine this region differs only by 3 amino acids. The zebrafish GDF-8 is more diverged, but it is still 88% identical to the human sequence in the carboxy-terminal region.

Because GDF-8 is a negative regulator of skeletal muscle mass, there is considerable interest in identifying and developing therapeutic methods involving factors that regulate the biological activity of GDF-8. For example, mice and cattle with mutations in the GDF-8 gene show a marked increase in body weight and muscle mass (McPherron et al., Nature 387:83-90 (1997); Zhu et al., FEBS Letters 474:71-75 (2000); Grobet et al., Nature Genet. 17:71-74 (1997)). Administration of a mouse monoclonal GDF-8 modulating antibody in the mdx mouse model of Duchenne muscular dystrophy (DMD), decreases muscle degeneration and serum creatine kinase concentrations, while increasing body weight, muscle mass, muscle size, and absolute muscle strength of the mdx mouse (Bodanovich et al., Nature 420:418-421 (2002)). Further, pharmacological inhibition of GDF-8 in adult C57BL/6 and BALB/c mice leads to an increase in muscle size and grip strength (Whittemore et al., BBRC 300:965-971 (2003)).

Due to its key function in the regulation of many critical biological processes, GDF-8 is a desirable target for therapeutic intervention for many disorders. Therapeutic agents that inhibit the activity of GDF-8 may be used to treat human or animal disorders in which an increase in muscle tissue would be therapeutically beneficial, and agents that modulate GDF-8 activity may be used to treat disorders associated with adipose tissue, glucose homeostasis, or a loss of bone. Further, a GDF-8 inhibitor administered to a normal individual, for example, may increase muscle mass in that individual.

One GDF-8 inhibitor is MYO-029, a fully human antibody which is described in further detail in U.S. Patent Pub. No. 2004-0142382. MYO-029 is capable of binding mature GDF-8 with high affinity, inhibiting GDF-8 activity in vitro and in vivo, and inhibiting GDF-8 activity associated with negative regulation of skeletal muscle mass. MYO-029 promotes increased muscle mass when administered to mice.

GDF-8 modulating agents are useful in a variety of therapeutic applications, and thus methods to detect and/or quantify GDF-8 modulating agents in a biological sample of an individual are desirable. Measurement of the levels of a therapeutic GDF-8 modulating agent in human serum has therapeutic importance. Such methods allow, for example, tracking the course of therapy, assessing pharmacokinetics or bioavailability of the agent, measuring the levels of an agent in a biological sample of an individual, and/or detecting administration of an agent that modulates GDF-8 activity.

Further, because inhibitors of GDF-8 activity developed for therapeutic applications increase muscle mass, they may be targets for abuse for performance enhancing purposes. The risk of illicit use of a GDF-8 modulating agent for non-therapeutic purposes rises as the agent becomes available as a therapeutic. Drugs administered to enhance athletic performance of an individual or to increase the growth rate or foodstuff properties of a livestock animal are, in many cases, regulated and/or banned. Thus, the ability to detect the abuse of GDF-8 modulating agents which have legitimate medical applications is increasingly important. It is therefore desirable to develop methods to detect the use of a GDF-8 inhibitor by an athlete or in a foodstuff animal, for example, and to monitor the use of a GDF-8 modulating agent in an individual.

A prior pharmacokinetic study of the GDF-8 modulating agent MYO-029 involved directly labeling the MYO-029 antibody with the radioactive isotope, ¹²⁵I. Direct detection is disadvantageous, however, as such methods may be cumbersome and may involve introducing potentially dangerous or toxic substances to the individual to whom the GDF-8 modulating agent is administered (U.S. Patent Pub. No. 2004/0142382-A1). Improved methods to detect a GDF-8 modulating agent in a biological sample are needed.

To monitor or assess therapy or to detect abuse of a GDF-8 modulating agent, it is therefore important to develop assays and methods to detect the presence of a GDF-8 modulating agent in a biological sample, and methods to monitor and/or quantitate a GDF-8 modulating agent in a biological sample.

### SUMMARY

Methods to detect a GDF-8 modulating agent in a biological sample, wherein the GDF-8 agent is able to modulate one or more GDF-8 activities, are described herein. Specifically, methods to detect GDF-8 inhibitors in biological samples are provided. These methods detect low levels of a GDF-8 modulating agent in a complex biological sample, such as serum, blood, plasma, or urine, for example. The methods may be used to detect various GDF-8 agents, and may be used for non-symptomatic, symptomatic, or healthy individuals, for example.

In one embodiment, a method to detect an exogenous GDF-8 modulating agent in a biological sample is provided, the method comprising: (a) adding a biological sample from an individual to be tested to an in vitro assay for a GDF-8 activity; (b) detecting modulation of the GDF-8 activity; and (c) comparing the modulation of the GDF-8 activity in the presence of the biological sample to the modulation of the GDF-8 activity in the presence of a control biological sample, thereby detecting the presence of the exogenous GDF-8 modulating agent in the biological sample.

In some embodiments, the in vitro assay comprises the steps of: (a) contacting a GDF-8 protein with a surface of a reaction vessel, wherein the GDF-8 protein is a mature GDF-8 protein dimer; (b) adding a biological sample to the reaction vessel; (c) adding a detection agent; and (d) detecting a GDF-8 modulating agent/GDF-8 protein complex associated with the surface of the reaction vessel, thereby detecting an exogenous GDF-8 modulating agent. In one embodiment, the GDF-8 protein comprises a biotin moiety and contacts the surface via the biotin moiety. In further embodiments, the GDF-8 is biotinylated on a lysine residue, the molar ratio of biotin moiety to GDF-8 protein is less than about 5:1, and/or the molar ratio of biotin moiety to GDF-8 protein is between about 0.5:1 and about 4:1. In other embodiments of this method, avidin or streptavidin is adsorbed to the surface of the reaction vessel prior to addition of the GDF-8 protein.

In an additional embodiment, the in vitro assay comprises the steps of: (a) contacting a soluble GDF-8 receptor with a surface of a reaction vessel; (b) adding a biological sample to the reaction vessel; (c) adding a labeled GDF-8 protein to the reaction vessel; and (d) detecting the amount of labeled GDF-8 protein/GDF-8 receptor complex associated with the surface in the presence and absence of the biological sample, wherein a reduction in the amount of labeled GDF-8 protein/GDF-8 receptor complex in the presence of the biological sample detects an exogenous GDF-8 modulating agent in the biological sample. In one embodiment, the method further comprises the step of incubating the biological sample with the labeled GDF-8 protein prior to adding the sample to the reaction vessel.

In still additional embodiments, the methods comprise a cell-based in vitro reporter gene assay that include the steps of: (a) providing a host cell comprising a reporter gene construct in a reaction vessel, wherein the construct comprises a GDF-8-responsive control element and a reporter gene; (b) adding a biological sample to the reaction vessel; and (c) detecting reporter gene expression in the cell in the presence and absence of the biological sample, thereby detecting an exogenous GDF-8 modulating agent.

In certain embodiments, the methods further comprise quantitating the level of the GDF-8 modulating agent in the biological sample by comparing the modulation of GDF-8 activity by the biological sample from an individual to a plurality of control samples, each comprising a known concentration of the GDF-8 modulating agent. In another preferred embodiment, the biological sample comprises a sample from an individual to whom a GDF-8 modulating agent has been or is suspected of having been administered. In other embodiments, the biological sample is chosen from serum, blood, plasma, biopsy sample, tissue sample, cell suspension, saliva, oral fluid, cerebrospinal fluid, amniotic fluid, milk, colostrum, mammary gland secretion, lymph, urine, sweat, lacrimal fluid, gastric fluid, synovial fluid, and mucus.

The methods provided herein may be used to detect a GDF-8 modulating agent chosen from, for example: an antibody that specifically binds to GDF-8; an antibody that specifically binds to a GDF-8 binding partner; a GDF-8 receptor; an ActRIIB protein; a follistatin-domain containing protein; a follistatin protein; a GASP-1 protein; a GDF-8 protein; a GDF-8 propeptide; a non-proteinacious inhibitor; and a small molecule. In certain embodiments, the GDF-8 modulating agent is a GDF-8 inhibitor. In other embodiments, the agent is an antibody that specifically binds to a GDF-8 protein. In one preferred embodiment, the GDF-8 modulating agent is MYO-029, a neutralizing human antibody that specifically binds to GDF-8.

In a further embodiment, a method to detect an exogenous GDF-8 modulating agent in a biological sample is provided that comprises: (a) contacting a mature GDF-8 protein with a surface of a reaction vessel; (b) adding a biological sample to the reaction vessel; (c) adding a detection agent to the reaction vessel; and (d) detecting a GDF-8 modulating agent/GDF-8 protein complex associated with the surface of the reaction vessel, thereby detecting the exogenous GDF-8 modulating agent in the biological sample. In preferred embodiments of this method, the mature GDF-8 protein comprises a biotin moiety and contacts the surface via the biotin moiety. In additional embodiments, the molar ratio of biotin moiety to GDF-8 protein is less than about 5:1 or the molar ratio of biotin moiety to mature GDF-8 protein is between about 0.5:1 and about 4:1.

GDF-8 modulating agents, such as GDF-8 inhibitors, may be detected in the methods provided herein, and they may also be used in the methods of the invention. Thus, in some embodiments, the detection agent is a GDF-8 inhibitor. In certain embodiments, the detection agent is chosen from an antibody that specifically binds to the GDF-8 modulating agent and a labeled GDF-8 protein. In additional embodiments, the detection agent is an antibody that specifically binds to the constant region of an immunoglobin, including a human immunoglobulin.

In other embodiments, a method to detect an exogenous GDF-8 modulating agent in a biological sample is provided that comprises: (a) contacting a capture agent with a surface of a reaction vessel, wherein the capture agent is chosen from a GDF-8 protein and a protein that specifically binds to a GDF-8 protein; (b) adding a biological sample to the reaction vessel; (c) adding a detection agent to the reaction vessel; and (d) detecting a GDF-8 modulating agent/capture agent complex associated with the surface of the reaction vessel, thereby detecting an exogenous GDF-8 modulating agent in the biological sample.

Further, a method to detect a GDF-8 modulating agent in a biological sample is provided that comprises: (a) contacting a GDF-8 receptor with a surface of at least a first and second reaction vessel; (b) adding a biological sample and a GDF-8 protein to the first reaction vessel; (c) adding a control sample and a GDF-8 protein to the second reaction vessel; (d) adding a detectable marker to the first and second reaction vessels; and (e) comparing the detectable marker signal in the first reaction vessel to the second reaction vessel, thereby detecting the GDF-8 modulating agent in the biological sample.

In another embodiment, a method to detect a GDF-8 modulating agent in a human biological sample is provided. This embodiment comprises (a) adding a biological sample to an in vitro assay for a GDF-8 activity; (b) detecting modulation of the GDF-8 activity; and (c) comparing the modulation of the GDF-8 activity in the presence of the test biological sample from the candidate to the modulation of the GDF-8 activity in the presence of a control biological sample, thereby detecting an exogenous GDF-8 modulating agent.

In a preferred embodiment, a method to detect MYO-029 in a biological sample is described, comprising: (a) contacting a biotinylated mature GDF-8 protein dimer with a surface of a reaction vessel, wherein the GDF-8 protein comprises a mean ratio of biotin to GDF-8 dimer of less than 5:1; (b) adding a biological sample to the reaction vessel; (c) adding a labeled antibody that specifically binds to a human immunoglobulin to the reaction vessel; and (d) detecting a MYO-029/biotinylated GDF-8 protein complex associated with the surface of the reaction vessel, thereby detecting exogenous MYO-029 in the biological sample.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

The foregoing summary and the following description are not restrictive of the invention as claimed.

### BRIEF DESCRIPTION OF THE SEQUENCES

DNA and amino acid (M) sequences of GDF-8, MYO-029, and relevant scFv fragments, V_{H} and V_{L} domains, and complementarity determining regions (CDR) are set forth in the Sequence Listing and are enumerated as listed in Table 1.

TABLE 1

| | SEQ ID NO |
|---|---|
| AA sequence of mature human GDF-8 | 1 |
| AA sequence of human GDF-8 precursor | 2 |
| DNA sequence of MYO-029 scFv | 3 |
| AA sequence of MYO-029 scFv | 4 |
| DNA sequence of MYO-029 V_{H} | 5 |
| AA sequence of MYO-029 V_{H} | 6 |
| DNA sequence of MYO-029 V_{L} | 7 |
| AA sequence of MYO-029 V_{L} | 8 |
| Germlined DNA seq. of MYO-029 scFv | 9 |
| Germlined AA seq. of MYO-029 scFv | 10 |
| Germlined DNA seq. V_{H} | 11 |
| Germlined AA seq. of MYO-029 V_{H} | 12 |
| Germlined DNA seq. of MYO-029 V_{L} | 13 |
| Germlined AA seq. of MYO-029 V_{L} | 14 |
| AA sequence of MYO-029 H1 | 15 |
| AA sequence of MYO-029 H2 | 16 |
| AA sequence of MYO-029 H3 | 17 |
| AA sequence of MYO-029 L1 | 18 |
| AA sequence of MYO-029 L2 | 19 |
| AA sequence of MYO-029 L3 | 20 |

### DETAILED DESCRIPTION

This invention relates to methods for detecting GDF-8 modulating agents in animals, including humans, that derive some benefit from modulation of at least one GDF-8 activity. Methods to detect the presence of exogenous GDF-8 modulating agent such as a GDF-8 inhibitor are provided herein. In particular, methods to assess the presence and/or quantity of a GDF-8 inhibitor in a biological sample from an individual to whom the GDF-8 inhibitor has been or is suspected of having been administered are provided.

When a GDF-8 modulating agent is administered to an individual, methods to detect the exogenous GDF-8 modulating agent are useful for determining the presence and/or quantity of the agent in a biological sample. The methods may also allow one to assess a therapeutic regimen, adjust the dosage of the agent, or assess the pharmacokinetics or bioavailability of the agent, for example.

In order that the present invention may be more readily understood, certain terms are first defined. Additional definitions are set forth throughout the detailed description.

The term “GDF-8” refers to a specific growth and differentiation factor-8. The term refers to the full-length unprocessed precursor form of GDF-8 as well as the mature and propeptide forms resulting from post-translational cleavage. Unless otherwise specified as “inactive,” a “GDF-8 protein” retains one or more GDF-8 biological activities. The term also refers to any fragments and variants of GDF-8 that maintain at least one biological activity associated with mature GDF-8, as discussed herein, including sequences that have been modified. The amino acid sequence of mature human GDF-8 is provided in SEQ ID NO:1, and the precursor, full-length human GDF-8 sequence is provided in SEQ ID NO:2. The present invention relates to GDF-8 from all vertebrate species, including, but not limited to, human, bovine, chicken, mouse, rat, porcine, ovine, turkey, baboon, and fish (for sequence information, see, e.g., McPherron et al., Proc. Nat. Acad. Sci. U.S.A. 94:12457-12461 (1997)).

The term “mature GDF-8” refers to the carboxy-terminal portion of the GDF-8 precursor protein. Depending on conditions, the mature GDF-8 may be present as a monomer, homodimer, and/or in a GDF-8 latent complex, for example. In its biologically active form, the mature GDF-8 is also referred to as “active GDF-8.” The term also refers to any fragments and variants of GDF-8 that maintain at least one biological activity associated with mature GDF-8, as discussed herein, including sequences that have been modified.

The term “GDF-8 propeptide” refers to the amino-terminal portion of the GDF-8 precursor protein. The GDF-8 propeptide is capable of binding to the propeptide binding domain on the mature GDF-8. The GDF-8 propeptide forms a complex with the mature GDF-8 homodimer. It is believed that two GDF-8 propeptides associate with two molecules of mature GDF-8 in the homodimer to form an inactive tetrameric complex, called a latent complex. The latent complex may include other GDF inhibitors in place of or in addition to one or more of the GDF-8 propeptides.

The term “GDF-8 activity” refers to one or more physiologically growth-regulatory or morphogenetic activities associated with active GDF-8 protein. For example, active GDF-8 is a negative regulator of skeletal muscle mass. Active GDF-8 can also modulate the production of muscle-specific enzymes (e.g., creatine kinase), stimulate myoblast proliferation, and modulate preadipocyte differentiation to adipocytes. “GDF-8 activity” includes “GDF-8 binding activity.” For example, mature GDF-8 specifically binds to the propeptide portion of GDF-8, to ActRIIB, to a GDF-8 receptor, to activin, to follistatin, to follistatin-domain-containing proteins, to GASP-1, and to other proteins. A GDF-8 inhibitor, such as an antibody or portion thereof, may reduce one or more of these binding activities. Exemplary procedures for measuring GDF-8 activity in vivo and in vitro are set forth below.

The term “GDF-8 modulating agent” includes any agent capable of modulating activity, expression, processing, or secretion of GDF-8, or a pharmaceutically acceptable derivative thereof. Agents that increase one or more GDF-8 activities and agents that decrease one or more GDF-8 activities are encompassed by the term. The term “GDF-8 inhibitor” includes any agent capable of affecting activity, expression, processing, or secretion of GDF-8, or a pharmaceutically acceptable derivative thereof. A GDF-8 inhibitor reduces one or more activities associated with GDF-8. In certain embodiments, a GDF-8 inhibitor will affect binding of GDF-8 to one or more of its physiological binding partners, including, but not limited to a receptor (e.g. ActRIIB), a follistatin-domain containing protein (e.g. follistatin, FLRG, GASP-1, GASP-2), or a GDF-8 protein such as the GDF-8 propeptide and mutants and derivatives thereof. Such GDF-8 inhibitors include, for example, antibodies that specifically bind to GDF-8 (including MYO-029, MYO-028, MYO-022, JA-16, and fragments and derivatives thereof), antibodies that specifically bind to a GDF-8 receptor, modified soluble receptors (including receptor fusion proteins, such as the ActRIIB-Fc fusion), other proteins that specifically bind to GDF-8 (such as the GDF-8 propeptide, mutants and derivatives of the GDF-8 propeptide, follistatin, follistatin-domain containing proteins, and Fc fusions of these proteins), proteins binding to the GDF-8 receptor and Fc fusions of these proteins, and mimetics are included. Nonproteinaceous inhibitors (such as nucleic acids) are also encompassed by the term GDF-8 inhibitor. GDF-8 inhibitors include proteins, antibodies, peptides, peptidomimetics, ribozymes, anti-sense oligonucleotides, double-stranded RNA, siRNA (e.g. for RNAi), and other small molecules, which specifically inhibit GDF-8. Such inhibitors are said to “inhibit,” “reduce,” or “neutralize” the biological activity of GDF-8, and are described in more detail below.

A GDF-8 inhibitor will. “inhibit”, “neutralize”, or “reduce” at least one biological activity of GDF-8, such as a physiological, growth-regulatory, or morphogenetic activity associated with active GDF-8 protein. For example, GDF-8 is a negative regulator of skeletal muscle growth. A GDF-8 inhibitor can increase muscle mass, increase muscle strength, modulate the levels of muscle-specific enzymes (e.g., creatine kinase), stimulate myoblast proliferation, modulate preadipocyte differentiation to adipocytes, decrease fat accumulation, decrease serum triglyceride levels, decrease serum cholesterol levels, modulate glucose metabolism, and/or reduce hyperglycemia.

The terms “inhibit,” “inhibitory,” and their cognates refer to a reduction in one or more activities of GDF-8 by a GDF-8 inhibitor, relative to the activity of GDF-8 in the absence of the same inhibitor. The reduction in activity is preferably at least about 20%, 30%, 40%, 50%, 60%, 70%, 80%, 90%, or higher. In certain embodiments, the activity of GDF-8, when affected by one or more of the presently disclosed inhibitors, is reduced at least 50%, preferably at least 60%, 62%, 64%, 66%, 68%, 70%, 72%, 74%, 76%, 78%, 80%, 82%, 84%, 86%, or 88%, more preferably at least 90%, 92%, 94%, 96%, 98% or 99%, and even more preferably at least 95% to 100%. The terms “neutralize,” “neutralizing,” and their cognates refer to a reduction one or more GDF-8 activities by at least 80%, 85%, 90%, or 95%. Inhibition of GDF-8 activity can be measured, for example, in pGL3(CAGA)₁₂ reporter gene assays (RGA) as described in Thies et al., Growth Factors 18:251-259 (2001) or in ActRIIB receptor assays as illustrated below.

The term “antibody,” as used herein, is any polypeptide comprising an antigen-binding site, such as an immunoglobulin or a fragment thereof, and encompasses any polypeptide comprising an antigen-binding site regardless of the source, species of origin, method of production, and characteristics. As non-limiting examples, the term “antibody” includes synthetic, human, orangutan, monkey, primate, mouse, rat, goat, dog, sheep, and chicken antibodies. The term includes but is not limited to polyclonal, monoclonal, monospecific, polyspecific, non-specific, humanized, single-chain, chimeric, synthetic, recombinant, hybrid, mutated, and CDR-grafted antibodies. For the purposes of the present invention, “antibody” also includes antibody fragments, unless otherwise stated (such as when preceded by the word “intact”). Exemplary antibody fragments include Fab, F(ab′)₂, Fv, scFv, Fd, dAb, and other antibody fragments that retain antigen-binding function. Typically, such fragments comprise an antigen-binding domain. As will be recognized by those of skill in the art, any of such molecules, e.g., a “human” antibody, may be engineered (for example “germlined”) to decrease its immunogenicity, increase its affinity, alter its specificity, or for other purposes.

Antibodies can be made, for example, via traditional hybridoma techniques (Kohler et al., Nature 256:495-499 (1975)), recombinant DNA methods (U.S. Pat. No. 4,816,567), or phage display techniques using antibody libraries (Clackson et al., Nature 352:624-628 (1991); Marks et al., J. Mol. Biol. 222:581-597 (1991)). For various other antibody production techniques, see Antibody Engineering (Borrebaeck ed., Oxford University Press 1995) and Antibodies: A Laboratory Manual, (Harlow et al., eds., Cold Spring Harbor Laboratory, 1988).

The term “antigen-binding domain” refers to the part of an antibody molecule that comprises the area specifically binding to or complementary to a part or all of an antigen. Where an antigen is large, an antibody may only bind to a particular part of the antigen. The “epitope” or “antigenic determinant” is a portion of an antigen molecule that is involved in specific interactions with the antigen-binding domain of an antibody. An antigen-binding domain may be provided by one or more antibody variable domains (e.g., an Fd antibody fragment consisting of a VH domain). In certain embodiments, an antigen-binding domain comprises an antibody light chain variable region (V_{L}) and an antibody heavy chain variable region (V_{H}) (U.S. Pat. No. 5,565,332).

The terms “specific binding,” “specifically binds,” or the like, mean that two or more molecules form a complex that is measurable under physiologic or assay conditions and is selective. An antibody or other inhibitor is said to “specifically bind” to a protein if, under appropriately selected conditions, such binding is not substantially inhibited, while at the same time non-specific binding is inhibited. Specific binding may be characterized by a relatively high affinity and is selective for the compound or protein. Nonspecific binding usually has a low affinity. Typically, the binding is considered specific when the affinity constant Kₐ is at least about 10⁶ M⁻¹, or preferably at least about 10⁷, 10⁸, 10⁹, or 10¹⁰ M⁻¹. Certain methods require high affinity for specific binding, whereas other methods, such as a surface plasmon resonance assay, may detect less stable complexes and lower affinity interactions. If necessary, non-specific binding can be reduced without substantially affecting specific binding by varying the binding conditions. Such conditions are known in the art, and a skilled artisan using routine techniques can select appropriate conditions. The conditions are usually defined in terms of concentration of the binding partners, ionic strength of the solution, temperature, time allowed for binding, concentration of non-related molecules (e.g., detergents, surficants, serum albumin, milk casein), etc. Exemplary binding conditions are set forth below.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure.

The term “individual” refers to any vertebrate animal, including a mammal, bird, reptile, amphibian, or fish. The term mammal includes any animal classified as such, male or female, including humans, non-human primates, monkeys, dogs, horses, cats, sheep, pigs, goats, cattle, etc. Examples of non-mammalian animals include chicken, turkey, duck, goose, fish, salmon, catfish, bass, frog, and trout. An individual may be chosen from humans, athletes, or domesticated, livestock, zoo, sports, racing, or pet animals, for example.

The term “effective dose,” or “effective amount,” refers to a dosage or level that is sufficient to ameliorate clinical symptoms of, or achieve a desired biological outcome (e.g., increasing muscle mass, muscle strength, and/or bone density) in individuals, including individuals having a GDF-8 associated disorder. Such amount should be sufficient to reduce the activity of GDF-8 associated with negative regulation of skeletal muscle mass and bone density, for example. Therapeutic outcomes and clinical symptoms may include reduction in body fat, increase in muscle mass, improved cardiovascular indicators, or improved glucose metabolism regulation. A GDF-8 inhibitor can increase muscle mass, increase muscle strength, increase body weight, modulate the levels of muscle-specific enzymes (e.g., creatine kinase), and/or stimulate myoblast proliferation, for example. In a preferred embodiment, a GDF-8 inhibitor reduces clinical manifestations of a GDF-8 associated disorder. A GDF-8 modulating agent can affect preadipocyte differentiation to adipocytes, decrease fat accumulation or body fat content, decrease serum triglyceride levels, decrease serum cholesterol levels, modulate glucose metabolism, modulate bone density, alter the ratio of muscle to fat in an individual, and/or reduce hyperglycemia, for example. A GDF-8 inhibitor may also be administered to an individual in order to increase muscle mass, to improve athletic performance, or to increase or accelerate growth, including muscle growth. The effective amount can be determined as described in the subsequent sections. A “therapeutically effective amount” of a GDF-8 inhibitor refers to an amount which is effective, upon single or multiple dose administration to an individual (such as a human) at treating, preventing, curing, delaying, reducing the severity of, or ameliorating at least one symptom of a disorder or recurring disorder, or prolonging the survival of the subject beyond that expected in the absence of such treatment.

A “GDF-8 associated disorder” is a disorder or condition in which a subject would benefit from the administration of a GDF-8 modulator, such as a GDF-8 inhibitor. GDF-8 associated disorders include medical disorders such as a muscle-related disorder, neuromuscular disorder, adipose tissue disorder, metabolic disorder, or bone-related disorder.

Administration of a GDF-8 inhibitor may be “therapeutic” when the inhibitor is administered to an individual to treat a disorder, which includes amelioration and/or prevention of symptoms or of the disorder. Therapeutic uses include the administration of a GDF-8 inhibitor to an individual having a medical disorder or who ultimately may acquire the disorder, in order to prevent, cure, delay, reduce the severity of, or ameliorate one or more symptoms of a disorder or recurring disorder, or in order to prolong the survival of a subject beyond that expected in the absence of such treatment. A GDF-8 inhibitor may also be administered to an individual in order to increase muscle mass, to improve athletic performance, or to increase or accelerate growth, including muscle growth. In the absence of the presence or risk of a medical disorder associated with GDF-8, such performance-enhancing methods for administering a GDF-8 inhibitor to an individual are generally deemed “non-therapeutic,” as herein defined.

A “biological sample” is biological material collected from an individual, such as cells, tissues, organs, fluids, and other clinical specimens and samples. Exemplary biological samples include serum, blood, and plasma.

The term “reaction vessel” refers to a container in which an association between a GDF-8 modulating agent and an antibody can occur and be detected. A “surface” is the outer part of any solid (such as, e.g., glass, cellulose, polyacrylamide, nylon, polystyrene, polyvinyl chloride, dextran sulfate, or treated polypropylene) to which a GDF-8 modulating agent can be directly or indirectly “contacted,” “immobilized,” or “coated.” A “surface of a reaction vessel” may be a part of the vessel itself, or the surface may be in the reaction vessel. A surface such as polystyrene, for example, may be subjected to chemical or radiation treatment to change the binding properties of its surface. Low binding, medium binding, high binding, aminated, and activated surfaces are encompassed by the term. A GDF-8 modulating agent can be directly contacted with a surface, e.g., by physical adsorption or covalent binding to the surface, or it can be indirectly contacted, e.g., through a interaction with a substance or moiety that is directly contacted with the surface.

The term “capture agent” as used herein, refers to a molecule, such as a protein, for example, that is used in an immunoassay to specifically bind to a target protein, such as a GDF-8 modulating agent or GDF-8 itself. A capture agent suitable for the instant methods specifically binds to the GDF-8 modulating agent and/or to GDF-8 protein. For example, a capture agent may be a GDF-8 protein, including a mature GDF-8 dimer, or a protein that specifically binds to a GDF-8 protein. Similarly, a capture agent may be a GDF-8 modulating agent or a protein that specifically binds to a GDF-8 modulating agent.

A “detection agent” is a protein or small molecule that allows detection of a GDF-8 modulating agent or a complex. In a preferred embodiment, the detection agent specifically binds to a GDF-8 modulating agent. A detection agent may optionally comprise a detectable label. A detection agent may also be itself detected by a substance comprising a detectable label. GDF-8 modulating agents detected by the methods provided herein, may also be used in the methods to detect other GDF-8 modulating agents, for example.

The term “label” refers to a molecule which, by its chemical nature, provides an analytically identifiable signal which allows the detection of a molecular interaction. A protein, including an antibody, has a detectable label if it is covalently or non-covalently bound to a molecule that can be detected directly (e.g., by means of a chromophore, fluorophore, or radioisotope) or indirectly (e.g., by means of catalyzing a reaction producing a colored, luminescent, or fluorescent product).

Methods to detect a GDF-8 modulating agent in a biological sample, wherein the GDF-8 modulating agent is able to modulate one or more GDF-8 activities are described herein. Specifically, methods to detect GDF-8 inhibitors in biological samples are provided. Methods are provided that encompass the detection of an exogenous GDF-8 modulating agent in a biological sample of an individual having or at risk for developing a GDF-8 associated disorder, or in a biological sample of a healthy individual who has potentially abused the same. The techniques provided herein are also able to detect or quantitate certain endogenous GDF-8 modulating agents, such as for the diagnosis of a GDF-8 associated disease.

These methods are especially suitable to detect low levels of a GDF-8 modulating agent in a complex biological sample, such as serum, blood, or plasma. The methods may be used to detect various GDF-8 modulating agents, and may be used in non-symptomatic, symptomatic, or healthy individuals, for example.

Exogenous GDF-8 Modulating Agents

A GDF-8 modulating agent, as provided herein, is capable of modulating activity, expression, processing, or secretion of GDF-8, or a pharmaceutically acceptable derivative thereof. A GDF-8 modulating agent may increase or decrease one or more GDF-8 activities. Agents that decrease one or more GDF-8 activities are GDF-8 inhibitors. While GDF-8 inhibitors are administered to increase muscle mass and to treat a muscle-related disorder or condition, a GDF-8 modulator, including a GDF-8 inhibitor, may be used to treat adipocyte disorders, glucose metabolism-related disorders, or bone disorders, for example. Naturally occurring mature GDF-8 dimer is expressly excluded from the definition of a GDF-8 modulating agent, as described herein. Variants and modified forms of GDF-8 that are altered from the native GDF-8 and that modulate a GDF-8 activity, however, are included within the meaning of the term GDF-8 modulating agent. This application is not intended to encompass detection of myostatin (GDF-8).

Biological derivatives of a GDF-8 modulating agent are encompassed by the term, such as modified forms of the agent that are present in a biological sample after administration of the agent to an individual. In certain embodiments, the methods to detect a GDF-8 modulating agent comprise methods that detect the presence of a GDF-8 modulating agent in a biological sample by assessing the presence of one or more biological derivatives, metabolites, or metabolic products of the GDF-8 modulating agent.

A GDF-8 modulating agent is “exogenous” if it is introduced from or produced outside of the organism from which the biological sample or biological material is obtained. An exogenous GDF-8 modulating agent may be directly introduced to an individual, such as by administration of the agent to the individual, or an exogenous GDF-8 modulating agent may be indirectly introduced to the organism. An exogenous GDF-8 modulating agent is indirectly introduced to an organism, for example, if it is administered in a precursor form, or if it is a protein that is synthesized within the organism from a DNA or RNA that was introduced to the animal or its ancestor.

Exogenous GDF-8 modulating agents may be differentiated from endogenous GDF-8 modulating agents by methods exploiting properties of the GDF-8 agent that are not present in endogenous factors according to methods that are disclosed herein and known in the art. For example, a GDF-8 modulating agent may be identified by its structure, affinity or activity. For instance, MYO-029, MYO-028, MYO-022, JA-16 and other monoclonal antibodies that specifically bind to a GDF-8 protein, comprise particular amino acid sequences and recognize one or more distinct epitopes of GDF-8. These agents may be identified by the addition of a labeled peptide epitope, for example a biotinylated peptide, that is specifically bound by the GDF-8 modulating agent. For example, peptide epitopes for MYO-029 are disclosed in U.S. Patent Pub. No. 2004/0142382 A1, and may be used to identify exogenous MYO-029 agent detected by a method provided herein. Similarly, peptide epitopes of JA-16 are set forth in U.S. Patent Pub. No. 2003/0138422 A1. Anti-idiotype antibodies may also be used to differentiate an exogenous antibody agent, for example. Also, antibodies specific to an exogenous GDF-8 modulating agent may be made by well known immunization or phage display techniques. MYO-029 specific antibodies are provided herein, as are methods of making the same, for example in Example 5.

Further, an exogenously administered agent may be distinguished from its naturally-occurring counterpart agent using fluorescence analysis (see, U.S. Pat. No. 6,680,207, for example). In addition, exogenous GDF-8 modulating agent may be distinguished from endogenous factors by the methods of U.S. Pat. No. 6,573,055, for example, which recognizes differences in glycosylation patterns based on the source cell type. A recombinantly produced biological product such as an antibody therapeutic (including MYO-029, MYb-028, MYO-022, or JA-16) or other glycosylated protein will comprise carbohydrate side chains, sugar chain structures, or glycopeptides that depend on the cell line or culture conditions in which the protein is produced. Monoclonal antibodies, polyclonal antibodies, peptide, nucleotide, or other substances that allow detection of a distinguishing feature of an exogenous GDF-8 modulating agent may also be used to identify an exogenous agent.

The GDF-8 modulating agents are detected by methods of the invention after administration of agent, including administration of an effective dosage of the agent. The agent may be administered at a dosage from about 50 ng/kg to about 20 mg/kg, including from about 2.5 mg/kg, depending on the severity of the symptoms and the progression of the disease, and may be as high as 200 mg/kg. A physician will select a dosage which is sufficient to reduce the activity of GDF-8 proteins to achieve a desired biological outcome, such as increasing skeletal muscle mass, increasing strength, or reducing one or more symptoms of the GDF-8 associated disease. Generally, a therapeutically-effective amount may vary with the subject's age, weight, physical condition, and sex, as well as the severity of the medical condition in the subject. The dosage may be determined by a physician and may also be determined by toxicity and therapeutic efficacy analyses using standard pharmaceutical procedures in cell cultures or experimental animals (e.g. LD₅₀, ED₅₀, therapeutic index) and adjusted, as necessary, to suit observed effects of the treatment. The appropriate effective dose is selected by a treating clinician from the following exemplary ranges: about 50 ng/kg to about 20 mg/kg, about 2.5 mg/kg to about 50 mg/kg, about 1 μg/kg to about 20 mg/kg, about 1 μg/kg to about 10 mg/kg, about 1 μg/kg to about 1 mg/kg, about 10 μg/kg to about 1 mg/kg, about 10 μg/kg to about 100 μg/kg, about 100 μg/kg to about 1 mg/kg, and about 500 μg/kg to about 5 mg/kg, about 1 mg/kg to about 10 mg/kg, and about 5 mg/kg to about 200 mg/kg. A single dose may be introduced, or dosing may be continuous, periodic or intermittent. Doses may be provided in daily, semi-weekly, weekly, bi-weekly, monthly, or bimonthly intervals, for example. The GDF-8 modulating agent to be detected is administered via topical, oral, intravenous, intraperitoneal, intramuscular, intracavity, subcutaneous or transdermal means, for example.

Because various GDF-8 modulating agents such as GDF-8 inhibitors may be used in the methods of the invention to detect a GDF-8 modulating agent, known GDF-8 inhibitors are described in further detail after description of the claimed methods. Further, as would be appreciated by one of skill in the art, detection agents used to detect the GDF-8 modulating agent vary with the structure of that agent. Thus, additional means to detect known GDF-8 modulating agents, including GDF-8 inhibitors, are provided with, and are apparent from, the detailed description of the same.

The present invention is directed to methods for detecting the presence of a GDF-8 modulating agent, and, more specifically, to methods to quantitate levels of GDF-8 modulating agents, including GDF-8 inhibitors, in a biological sample of an individual. The methods are especially suitable for use for evaluating the course of therapy with a GDF-8 modulating agent, assessing pharmacokinetics or bioavailability of the agent, measuring the levels of an agent in a biological sample of an individual, and/or to detecting administration of an agent that modulates GDF-8 activity to an individual. In one embodiment, the methods detect the presence in a biological sample of MYO-029, a neutralizing monoclonal antibody that specifically binds to GDF-8.

Identification of Candidates

An individual receiving treatment for a GDF-8 associated disorder with a GDF-8 modulating agent is a candidate for the methods herein provided to detect exogenous GDF-8 modulating agent in a biological sample of the individual. Further, an individual with a GDF-8 associated disorder, or an individual at risk for developing a GDF-8 associated disorder or a muscle-related disorder, may be a candidate for the methods provided herein.

In certain embodiments, an individual who is identified as receiving a GDF-8 modulating agent, for example in an effective amount, will be a candidate for the methods herein. An individual undergoing therapy with a GDF-8 modulating agent may have levels of the GDF-8 modulating agent that change during a course of therapy, thereby impacting the efficacy of the treatment. Further, prior to treatment of a GDF-8 associated disorder with a GDF-8 modulating agent, suitable candidates for administration of a GDF-8 modulating agent may be identified with the methods provided herein, as it may be desirable to detect and control for individual variations in drug clearance or bioavailability associated with the administration of a GDF-8 modulating agent.

An individual having, or at risk for developing, a muscle-related disorder is a candidate for the methods provided herein. Inhibition of GDF-8 activity increases muscle tissue in individuals, including those suffering from muscle-related disorders. A number of disorders are associated with functionally impaired muscle tissue, e.g., muscular dystrophies, amyotrophic lateral sclerosis (ALS), muscle atrophy, organ atrophy, frailty, congestive obstructive pulmonary disease, heart failure, sarcopenia, cachexia, and muscle wasting syndromes caused by other diseases and conditions.

A muscle-related disorder includes, for example, muscular dystrophies, amyotrophic lateral sclerosis (ALS), sarcopenia, cachexia, muscle wasting, muscle atrophy, or muscle degeneration, including wasting, atrophy, or frailty. Muscular dystrophies include, for example, pseudohypertrophic, facioscapulohumeral, and limb-girdle muscular dystrophies. Exemplary muscular dystrophies include Duchenne's muscular dystrophy (Leyden-Mbbius), Becker muscular dystrophy, Emery Dreifuss muscular dystrophy, limb girdle muscular dystrophy, rigid spine syndrome, Ullrich syndrome, Fukuyama muscular dystrophy, Walker Warburg syndrome, muscle eye brain disease, facioscapulohumeral muscular dystrophy (Landouzy-Dejerine), congenital muscular dystrophy, myotonic dystrophy (Steinert's disease), myotonia congenital, and Gowers disease. Muscle degeneration associated with or secondary to another disease or condition such as cardiovascular disease, organ atrophy, organ failure, cancer, Acquired Immune Deficiency Syndrome (AIDS), bed rest, immobilization, prolonged lack of use, or other disease or condition is also included in the term.

Individuals with muscle-loss or muscle wasting associated with cardiovascular disorders are also candidates for the methods provided herein. Examples of cardiovascular disorders include coronary artery disease (atherosclerosis), angina (including acute angina and unstable angina), heart attack, stroke (including ischemic stroke), hypertension associated cardiovascular diseases, heart failure, congestive heart failure, coronary artery disease, hypertension, hyperlipidemia, peripheral arterial disease, and peripheral vascular disease. Examples of disorders of insulin metabolism include conditions associated with aberrant glucose homeostasis, type 2 diabetes, prediabetes, impaired glucose tolerance, dyslipidemia, metabolic syndrome (e.g., syndome X), and insulin resistance induced by trauma such as burns or nitrogen imbalance.

An individual having, or at risk for developing, an adipose tissue, metabolic, or bone-related disorder or condition is also a candidate for a method as claimed. Such disorders or conditions include those associated with glucose homeostasis such as, e.g., development of type 2 diabetes, impaired glucose tolerance, metabolic syndromes (e.g., syndrome X), insulin resistance induced by trauma, such as burns or nitrogen imbalance, and adipose tissue disorders (e.g., obesity) (Kim et al., Biochem. Biophys. Res. Comm. 281:902-906 (2001)). For example, GDF-8 modulates preadipocyte differentiation to adipocytes (Id.) and inhibits adipocyte formation from mesenchymal precursor cells and preadipocytes (Rebbapragada et al., Mol. Cell Bio. 23:7230-7242 (2003)). Fat accumulation is reduced both in GDF-8 knock-out mice and in wild-type adult mice in which GDF-8 protein has been systematically administered (McPherron et al., J. Clinical Invest. 109:595-601 (2002); Zimmers et al., Science 296:1486-1488 (2002)). Disorders or conditions assosicated with bone loss include osteoporosis and osteoarthritis, especially in the elderly and/or postmenopausal women, glucocorticoid-induced osteoporosis, osteopenia, osteoarthritis, and osteoporosis-related fractures. In addition, metabolic bone diseases and disorders characterized by low bone mass are included, such as those due to chronic glucocorticoid therapy, premature gonadal failure, androgen suppression, vitamin D deficiency, secondary hyperparathyroidism, nutritional deficiencies, and anorexia nervosa.

Further, an individual exhibiting an increase in muscle mass, such as an increase in muscle cell size (hypertrophy) or muscle cell number (hyperplasia) may be a candidate for a method to detect exogenous GDF-8 modulating agent. The increase can be in type 1 and/or type 2 muscle fibers of a mammal or other animal. Methods to measure an increase in muscle mass are well known in the art. For example, muscle can be measured before and after administration of a GDF-8 modulating agent using standard techniques such as underwater weighing. An increase in muscle size may be evidenced by weight gain of at least about 5%, 10%, 20%, or more. Other non-invasive technologies may be used, including magnetic resonance imaging (MRI) or dual-energy X-ray absorptiometry (DEXA) technology, for example. Athletes, including professional athletes, are candidates for the methods.

An individual who has taken or who is suspected of taking a GDF-8 modulating agent such as, for example, MYO-029, for performance enhancing reasons is a candidate for these methods. In other embodiments, an individual such as a cow or other livestock animal is a candidate for a method provided herein, when it may be desirable to detect the administration of an exogenous GDF-8 modulating agent in a biological sample of such an animal. For example, an exogenous agent may be administered to increase growth or muscle tissue mass (or to reduce the fat content of meat) in livestock animals.

In a first embodiment, a method to detect an exogenous GDF-8 modulating agent in a biological sample is provided, the method comprising: adding a test biological sample from an individual to an in vitro assay for a GDF-8 activity, detecting modulation of the GDF-8 activity, and comparing the modulation of the GDF-8 activity in the presence of the test biological sample to the modulation of the GDF-8 activity in the presence of a control biological sample, thereby detecting the presence of the exogenous GDF-8 modulating agent in the biological sample. In certain embodiments, the methods further comprise quantitating the level of the GDF-8 modulating agent in the biological sample by comparing the modulation of GDF-8 activity by the test biological sample to a plurality of control samples, each comprising a known concentration of the GDF-8 modulating agent.

In certain embodiments, the in vitro assay measures one or more physiologically growth-regulatory or morphogenetic activities associated with active GDF-8 protein. In vitro assays to detect modulation of a GDF-8 activity are well known in the art, and may be chosen from a cell-based assay or cell-free assay (such as, e.g., an assay to measure modulation of transcription, replication or cell cycle arrest) or a binding assay (such as, e.g., an immunoassay, a surface plasmon resonance assay, immunoprecipitation, or a radioimmune assay). For example, active GDF-8 is a negative regulator of skeletal muscle mass, it modulates the production of muscle-specific enzymes (e.g., creatine kinase), stimulates myoblast proliferation, and modulates preadipocyte differentiation to adipocytes. In some methods, selection of GDF-8 modulating agents from BMP-11 modulating agents is performed. Cell-based and cell free assays for a GDF-8 activity are known in the art and are described infra.

A biological sample, such as a test biological sample, comprises biological material from at least one individual. In preferred embodiments, the individual is undergoing therapy with a GDF-8 modulating agent. In other preferred embodiments, the individual is a candidate for administration of a GDF-8 modulating agent. In further embodiments, the individual is an mammal, bird, reptile or fish. In particular embodiments, the biological sample is chosen from serum, blood, plasma, biopsy sample, tissue sample, cell suspension, saliva, oral fluid, cerebrospinal fluid, amniotic fluid, milk, colostrum, mammary gland secretion, lymph, urine, sweat, lacrimal fluid, gastric fluid, synovial fluid, and mucus. In preferred embodiments, the biological sample is a fluid. In some preferred embodiments, the biological sample is chosen from blood, serum, and plasma. In specific embodiments, the biological sample is serum, such as human, primate, monkey, rat or mouse serum.

In other embodiments, the biological sample is isolated from an individual or individuals and optionally treated prior to testing. For example, the biological sample may also be used as collected or after dilution with a suitable diluent. Dilutions are optimized to reduce and/or eliminate matrix interference with the assay. The diluent is not particularly restricted but may comprise serum, including e.g., human serum, deionized water or various buffers having a buffer action within the range of about pH 5 to about pH 9, preferably about pH 6.5 to about pH 8.5, (e.g. citrate buffer, phosphate buffer, Tris buffer, acetate buffer, or borate buffer). In some preferred embodiments, the diluent comprises normal human serum. The diluent may comprise a constant concentration of a control biological sample, e.g. to reduce variability due to matrix effects with increasing dilution of the test biological sample.

The dilution buffer may optionally comprise a constant amount of a control biological sample, chosen to correspond to the test biological sample, for example to control for background effects or interference of the sample matrix. In one embodiment, a test sample of human serum is diluted in THST buffer (300 μL/well) (50 mM Tris-HCl, pH 8.0, containing 1.0 mM glycine, 0.5 M NaCl, and 0.05% v/v/ Tween 20® (J. T. Baker)) 1:8 fold, and dilutions of the test sample beyond 8-fold are prepared in THST plus 12.5% human serum. Also, a sample may be diluted approximately 2, 4, 8, 16, 32, 64, or 128-fold or higher. In other embodiments, a test sample is serially diluted 1:1.5 or 1:1.6 to obtain a range of data points that allow verification of dilutional linearity and matrix effects. For preferred biological sample matrices, a dilution may be selected at which conditions related to matrix interference and assay sensitivity are optimized.

In some embodiments, the sample may be optionally fractionated or concentrated using well known methods and then added to a method provided herein to detect a GDF-8 modulating agent. Fractionation (including purification) or concentration may be used, for example, if matrix interference limits detection of a GDF-8 modulating agent in the assay. Fractionation and concentration techniques, include, but are not limited to, centrifugation, ammonium sulfate precipitation, polyethylene glycol precipitation, trichloroacetic acid (TCA) precipitation, affinity techniques (such as immunoprecipitation with a resin conjugated to a specific binding partner such as an antibody, i.e., an anti-human Fc antibody, protein A or protein G, for example), chromatographic techniques, and other separation techniques. In preferred embodiments, the biological sample is not fractionated or concentrated prior to detection of a GDF-8 modulating agent.

A biological sample may be collected from a naïve individual, or a sample may be taken before, during or after administration of a GDF-8 modulating agent. For example a sample may be obtained from an individual 1, 2, 4, 6, 8, 10, 12, 15, 20, 25, 30, or more days after administration of a GDF-8 modulating agent. A sample may also be obtained 1, 2, 3, 4, 6, 8, 10, 12, 16, or more weeks after administration of a GDF-8 modulating agent. The timing of sample collection may be optimized to increase detection of a GDF-8 modulating agent, or to detect altered bioavailability of the agent.

The GDF-8 modulating agent detected by the methods provided herein may be an antibody that specifically binds to a GDF-8 protein, and in a preferred embodiment, the GDF-8 modulating agent is MYO-029. In certain embodiments, the GDF-8 modulating agent to be detected is chosen from: an antibody, an antibody that specifically binds to GDF-8; an antibody that specifically binds to a GDF-8 binding partner, a GDF-8 receptor, an ActRIIB protein, a follistatin-domain containing protein, a follistatin protein, a GASP-1 protein, a GDF-8 protein, a GDF-8 propeptide, a non-proteinacious inhibitor, and a small molecule (described in further detail above).

As would be readily apparent to one of skill in the art, the GDF-8 modulating agent is detectable with a detection agent that is selected based on the in vitro assay and the GDF-8 modulating agent to be detected (see below). Where the in vitro assay is a reporter gene assay, the detection agent is preferably a reporter gene product, such as an enzyme or protein comprising a label such as an epitope tag. Suitable enzymes include peroxidase (e.g., horseradish peroxidase), alkaline phosphatase, glucose oxidase, β-galactosidase, and other proteins capable of catalyzing a reaction to produce a colored, luminescent, or fluorescent product, for example. Where the in vitro assay is a binding assay, such as, for example, an enzyme-linked immunosobant assay (ELISA), the detection agent will differentially associate with a capture protein and a capture protein in complex with the GDF-8 modulating agent detected by the methods provided herein. A detection agent may be a protein, e.g. an antibody, that specifically binds to a GDF-8 modulating agent or to a GDF-8 modulating agent:capture protein complex. Alternatively, a detection agent may be a protein that affects the binding of the GDF-8 modulating agent to the capture protein.

Reporter Gene Assay

In one aspect, the in vitro assay is a reporter gene assay (RGA) (see, Thies et al., Growth Factors 18:251-259 (2001)). In certain embodiments, an RGA comprises the steps of: (a) providing a host cell comprising a reporter gene construct in a reaction vessel, wherein the construct comprises a GDF-8-responsive control element and a reporter gene; (b) adding a biological sample to the reaction vessel; and (c) detecting reporter gene expression in the cell in the presence and absence of the biological sample, thereby detecting an exogenous. GDF-8 modulating agent. In certain embodiments, the method comprises the further step of adding a substrate that changes color, luminescence, or fluorescence in the presence of the reporter gene.

A host cell may be a eukaryotic cell, such as from a human, mammal, or other animal. In a preferred embodiment, the host cell is a cell line, such as a eukaryotic cell line, a mammalian cell line, or a cancer cell line, including a rhabdosarcoma cell line. The reporter gene construct may be transiently or stably introduced into the host cell by any means known in the art, including transfection, electroporation, and the like. The reporter gene construct comprises a GDF-8-responsive control element (such as promoter and/or enhancer sequences), and a reporter gene in operative association with the control element (see, for example U.S. Patent Pub. No. 2003/0138422, and references described therein).

For example, to demonstrate the activity of GDF-8, a reporter gene assay has been developed using a reporter vector pGL3(CAGA)₁₂ expressing luciferase. The amount of GDF-8 protein added to the assay may be titrated for optimization. An amount of GDF-8 protein is selected that is sufficient to produce 40%, 50%, 60%, 70%, 80%, or 90% of maximal reporter construct activation. GDF-8 protein may be added at 0.5, 1, 5, 10, 20, 30, 40, 50, 60, 70, 80, 90, 100, 150, or 200 ng/mL, for example. Using a constant amount of GDF-8 protein, the GDF-8 modulating agent may be titrated to prepare a control titration of modulation of GDF-8 activity. For example, a GDF-8 modulating agent such as MYO-029 may be tested at concentrations selected from 0.05, 0.1, 0.5, 1, 5, 10, 20, 30, 40, 50, 60, 70, 80, 90, 100, 150, 200, 500, or 1,000 ng/mL, for example. In preferred embodiments, a GDF-8 modulating agent titration will span the linear range of inhibition in the assay.

Cells are then treated with or without 10 ng/mL GDF-8, for example, and with or without the test biological sample in McCoy's 5A media with glutamine, streptomycin, penicillin, and 1 mg/mL bovine serum albumin for 6 hrs at 37° C. In certain embodiments, known GDF-8 modulating agent controls are run in parallel using concentrations from 10 pM to 50 μM, approximately. Exemplary concentrations include 10 pM, 50 pM, 100 pM, 1 nM, 10 nM, 50 nM, 100 nM, 500 nM, 1 μM, 5 μM, 10 μM, and 50 μM of GDF-8 modulating agent. In preferred embodiments, the amount of GDF-8 modulating agent in the test sample is compared to a control titration of known amounts of the agent, and thereby quantitated. Reporter gene protein, such as an enzyme that catalyzes the conversion of a substrate to a colorimetric, fluorescent or luminescent molecule may be quantified in the treated cells using well known techniques.

Binding Assays

In certain embodiments, the in vitro assay measures a GDF-8 binding activity. An in vitro assay may detect an exogenous GDF-8 modulating agent that binds to a GDF-8 protein or an agent that binds to a GDF-8 binding partner, such as a protein that specifically binds to GDF-8. For example, mature GDF-8 specifically binds to the propeptide region of GDF-8, to ActRIIB, to a GDF-8 receptor, to follistatin, to follistatin-domain-containing proteins, to GASP-1, and to other proteins. In a particular embodiment, a GDF-8 modulating agent such as an antibody or portion thereof, reduces one or more of these binding activities and this effect on binding is detected. In certain embodiments, the specific binding of a GDF-8 modulating agent to a GDF-8 protein, for example, is detected. In some cases, a capture protein for an in vitro binding assay is chosen from a GDF-8 protein or a protein that specifically binds to GDF-8. In certain embodiments, the binding of a GDF-8 modulating agent to the capture protein is measured in an ELISA. In some embodiments, the binding of the capture protein to a second protein (such as GDF-8) is measured in the presence and absence of the test biological sample. The binding may be observed with a detection agent. In certain preferred embodiments, detection comprises surface plasmon resonance technology, optionally including surface plasmon fluorescence spectroscopy (SPFS), for example with surface plasmon spectroscopy (SPS). Detecting fluorescence intensity of a labeled molecule, for example a fluorescently labeled detection agent, in addition to the SPS reflectivity, improves the sensitivity in certain embodiments involving SPS detection. Standard SPS procedures are also included. In some embodiments, ELISAs are performed, including assays in a direct-binding assay format, a bridge format (in which the GDF-8 modulating agent would simultaneously bind solid phase GDF-8 and e.g., fluid-phase biotinylated GDF-8, for example), or in a competitive format.

In a binding assay, a detection agent will recognize and bind to the exogenous GDF-8 modulating agent, for example, and can be used alone or in combination with other reagents to generate a practicable dose-response signal that may be utilized to detect inhibitors of GDF-8, for example. In certain embodiments, the detection agent used to detect a GDF-8 modulating agent is specific for a particular GDF-8 modulating agent or group of GDF-8 modulating agents. For example, in a preferred embodiment, an antibody that specifically binds to a human immunoglobulin sequence is used to detect the human antibody-based GDF-8 modulating agent, MYO-029.

In a specific example, see, e.g., Example 1, the preferred detection reagent is a mouse anti-human IgG-horseradish peroxidase conjugate; however, any reagent capable of recognizing and binding to human IgG generally, or to human IgG1 with lambda light chains, or to the idiotype or allotype of MYO-029 specifically, could be used as a basis for detection of MYO-029.

In other embodiments, such as when the in vitro assay measures competitive binding (e.g. a competitive ELISA), a detection agent may be a labeled GDF-8 protein, including a biotinylated mature GDF-8 dimer. A labeled GDF-8 protein may also be the detection agent, for example, to detect a GDF-8 modulating agent (such as, for example, the MYO-029 antibody) that comprises one or more GDF-8 binding moieties.

In other embodiments, a detection agent is an antibody that specifically binds to the GDF-8 modulating agent. In some instances, a detection agent is an antibody that specifically binds to mature GDF-8, such as MYO-029, MYO-028, MYO-022, or JA-16. In embodiments in which the GDF-8 modulating agent is a human antibody, the detection agent may be an antibody that specifically binds to the GDF-8 modulating agent, such as an anti-human Ig, including a mouse anti-human Fc antibody. In an ELISA, the complex will be detected with an enzyme label.

Immunoassays

In one embodiment, the present invention comprises a binding assay in which a GbF-8 protein, such as mature GDF-8 dimer or other capture agent, is contacted with a surface of a reaction vessel, a biological sample is added, and a detection agent is added, thereby detecting an exogenous GDF-8 modulating agent in the biological sample.

More specifically, the present invention comprises a method for detecting an exogenous GDF-8 modulating agent in a biological sample such as serum, which comprises the following steps: (a) contacting a capture agent with a surface of a reaction vessel, (b) adding a biological sample to the reaction vessel, (c) adding a detection agent to the reaction vessel, and (d) detecting a GDF-8 modulating agent/capture agent complex associated with the surface of the reaction vessel.

In step (a) the capture protein, such as GDF-8 protein, is immobilized on the solid surface of a reaction vessel, for example by being either covalently or non-covalently bound to the surface. The solid surface is typically glass or a polymer, such as, e.g., cellulose, dextran sulfate, polyacrylamide, nylon, polystyrene, polyvinyl chloride or polypropylene and may be in the form of a bead, including a magnetic or paramagnetic bead. Immobilization of the ligands on the surface can be achieved by covalent bonding or by non-covalent interactions, such as physical adsorption. Covalent bonding methods include coupling with a crosslinking agent such as glutaraldehyde, hexamethylene isocyanate, a sulfo-containing agent, a peptide, an alkylating agent, or a similar reagent. In preferred embodiments, the GDF-8 is a mature GDF-8 dimer. In other preferred aspects, the mature GDF-8 protein is biotinylated and the surface of the reaction vessel is coated (e.g., via adsorption) with avidin or streptavidin. In certain embodiments, the methods provided herein comprise use of a biotinylated mature GDF-8 dimer that retains at least one GDF-8 activity.

These methods arise from the discovery that biotinylated mature GDF-8 is a more effective capture agent than mature GDF-8 protein adsorbed to a surface of a reaction vessel. Further, mature GDF-8 is unexpectedly sensitive to biotinylation of primary amine groups, such as on lysine residues. Hyperbiotinylated GDF-8, when biotinylated with amine specific biotinylation reagents, is less active or inactivated as compared to GDF-8 without biotin. The number of biotin moieties incorporated on amine groups per mature GDF-8 dimer was found to be critical for preparations that retain GDF-8 activity. For example, MYO-029 and ActRIIB binding activities are reduced in hyperbiotinylated preparations. Therefore, amine biotinylated mature GDF-8 preparations having less than five moles of biotin per mole of GDF-8 dimer are preferred. In alternate embodiments, proteins may be biotinylated on sulfhydryls, carboxyls, and/or carbohydrates. Photoreactive biotin compounds that non-specifically bind or react upon photoactivation are also available.

In certain methods provided herein, GDF-8 is biotinylated an amine-specific biotinylation reagent as a latent complex, and subsequently mature GDF-8 is isolated from the complex. In these methods, the amount of biotin incorporated into the mature GDF-8 dimer is optimized to retain biological activity, for example to avoid inactivating the receptor binding site. GDF-8 protein may also be biotinylated on surface cysteine residues (or surface thiol groups) using a sulfhydryl-specific biotinylation reagent. Additionally, methods to biotinylate carbohydrates involving oxidative pretreatment to generate reactive aldehydes and the use of biotin hydrazide reagents, for example, are known in the art and may be optimized for proteins described herein, including for mature GDF-8 protein, optimally in modified form. Further, carbbxyl reactive biotinylation reagents and reactions that allow biotinylation via aspartate and glutamate residues, for example, may be used. As would be apparent to one of skill in the art, the optimal molar ratios of biotin to GDF-8 dimer will vary with the biotinylation procedure and reagent utilized. For example, a skilled artisan will appreciate how to optimize an active biotinylated GDF-8 preparation using the methods described herein in combination with known biotinylation procedures, to produce a biotinylated mature GDF-8 protein that has different optimal molar ratios of biotin to GDF-8 dimer, while retaining at least one GDF-8 activity.

In some embodiments, mature GDF-8 protein is biotinylated with amine-specific biotinylation reagents. For example, GDF-8 preparations may be biotinylated on lysine residues and/or amino termini. Functional, mature GDF-8 protein may be biotinylated as part of a latent complex, and subsequently mature GDF-8 is isolated from the complex, e.g. as set forth in Example 3. In an alternative preparation, GDF-8 protein in the latent complex is produced and isolated according to the assay of Example 1 of U.S. Patent Pub. No. 2004/0142382 A1. The latent complex is subsequently biotinylated using well known techniques and/or as described herein.

Various biotinylation reagents are capable of efficient labeling of proteins, including a GDF-8 latent complex. Molar ratios of biotin derivative to GDF-8 latent complex in the reaction may be about 10, 15, 20, 40, or 80 to 1, and reagent composition and concentration reaction times, and temperatures may be varied to adjust the amount of biotin incorporated in the reaction. For example; salts and other agents may optionally be optimized. In an embodiment, the mature GDF-8 dimer is biotinylated in association with the amino terminal propeptide portion of GDF-8 to avoid inactivating the mature dimer during the biotinylation reaction.

Biotin derivatives are well known and available in the art. Modifications of biotin include variable spacer arms, modifications to affect solubility, and/or reactive groups, for example, to allow cleavage of the biotin moiety. Succinimidyl esters of biotin and its derivatives, including water soluble sulfosuccinimidyl esters may be used for biotinylation of GDF-8 on lysine residues, for example. To quantitate the amount of biotin incorporated, for example, well known analytical and sizing techniques are used, including reverse phase high pressure liquid chromatography, mass spectroscopy, etc. Additionally, commercial kits for quantitating biotin by colorimetric or fluorimetric assays, for example, are available (see, e.g., EZ™ Biotin Quantitation Kit, Pierce, utilizing HABA (2-(4′-hydroxyazo benzene)-benzoic acid)).

A further exemplary biotinylation procedure includes biotinylating GDF-8 latent complex at a ratio of 15 or 20 moles of EZ-Iink Sulfo-NHS-Biotin (Pierce, Cat. No. 21217) to 1 mole of the GDF-8 complex for 2 hours at 2-8° C. (see, for example, Example 3 of U.S. Patent Pub. No. 2004/0142382 A1). The reaction may be terminated by dropping the pH using 0.5% TFA and then the complex is subjected to chromatography on a C4 Jupiter 250×4.6 mm column (Phenomenex), separating mature GDF-8 from GDF-8 propeptide. Biotinylated mature GDF-8 fractions eluted with a TFA/CH₃CN gradient are pooled, concentrated and quantified by MicroBCA™ protein Assay Reagent Kit (Pierce, Cat. No. 23235), or using other well known isolation and concentration techniques.

In a preferred embodiment, an in vitro binding assay comprises a biotinylated GDF-8 protein capture agent, and the GDF-8 protein contacts the surface of the reaction vessel through interaction of the biotin moiety with avidin on the surface of the reaction vessel. In some embodiments, the molar ratio of biotin moiety to mature GDF-8 protein is between about 0.5:1 and about 4:1 In other embodiments, the mean ratio of biotin to GDF-8 dimer is less than about 5 to 1, less than about 2 to 1, or less than about 1 to 1. The ratio of biotin to mature GDF-8 protein has been measured to be a mixture of molar ratios of 0 to 3 in active GDF-8 preparations, with the majority of the molecules being at about 1:1. The mode for the ratio of biotin to mature GDF-8 protein may be less than or approximately 1, 2, 3, 4, or 5, for example. In some embodiments, the biotinylated mature GDF-8 preparation includes less than about 1, 2, 3, 4, or 5 moles of biotin per mole of mature GDF-8 dimer. The mean or median ratio of biotin to mature GDF-8 protein may be less than or approximately, 0.5, 0.75, 1, 1.25, 1.5, 2, 2.5, 3, 3.5, 4, 4.5, 5, 5.5, 6, 7, 8, or 9, for example. Other detection and capture agents may also be labeled by biotinylation. For example, biotinylated MYO-029 may be biotinylated up to a ratio of at least (or less than) 10:1, 20:1, or higher, for example. Optionally, another capture agent may be used.

After contacting the capture agent to the surface of the reaction vessel, the reaction vessel is washed to remove unattached capture agent prior to addition of the biological sample. In some embodiments the reaction vessel is washed with a buffer with pH between about 5 and about 9, such as citrate buffer, phosphate buffer, Tris buffer or acetate buffer. Optionally, detergent concentration or ionic strength may be added. Non-specific interactions are minimized with a blocking step, wherein a buffer comprising at least one blocking agent, such as a protein that does not specifically bind to the target is added to the reaction vessel. In other embodiments, detergents may be added, such as ionic or non-ionic detergents. Blocking buffers may comprise serum, bovine serum albumin, milk, casein, gelatin, and/or non-ionic detergents, for example. In some embodiments the reaction vessel is washed with a buffer with pH between about 5 and about 9, such as citrate buffer, phosphate buffer, Tris buffer or acetate buffer.

In step (b) a biological sample is added to the reaction vessel. In some preferred embodiments of the invention, the biological sample is chosen from blood, serum, and plasma. The biological sample may be used as collected or after dilution with a suitable diluent. The diluent is not particularly restricted but includes deionized water and various buffers having a buffer action within the range of about pH 5 to about pH 9, preferably about pH 6.5 to about pH 8.5, (e.g. citrate buffer, phosphate buffer, Tris buffer, acetate buffer, or borate buffer).

An aliquot of the sample to be tested is contacted with the immobilized capture agent and incubated for a period of time sufficient (e.g., 2-120 minutes) and under suitable conditions (e.g., 23° C.) to allow binding of a GDF-8 modulating agent present in the sample to the immobilized protein, such as biotinylated mature GDF-8 dimer. The GDF-8 modulating agent/GDF-8 protein reaction is not particularly restricted but can be conducted under the conditions in routine use for conventional immunoassays. A typical procedure comprises incubating or allowing to stand a reaction system comprising the detection agent and GDF-8 modulating agent generally at a temperature of not over 45° C., preferably between about 4° C. and about 40° C., more preferably between about 20° C. and about 40° C., or for between about 0.5 and 24 hours, preferably between about 1 and about 2 hours. The solvent is not particularly restricted provided that it does not interfere with the reaction, and thus includes, but is not limited to, buffers at between about pH 5 and about pH 9, such as citrate buffer, phosphate buffer, Tris buffer and acetate buffer. Detergents may optionally be present.

Step (c) comprises adding a detection agent to the reaction vessel. Following the incubation period, the immobilized GDF-8 modulating agent/capture agent complex is, in some embodiments, washed with buffer to remove unbound solutes before step (c). In other embodiments a simultaneous assay is performed, whereby steps (b) and (c) occur concurrently.

When step (c) is conducted after step (b), a typical procedure comprises incubating or allowing to stand a reaction system comprising the detection agent and the GDF-8 modulating agent generally at a temperature of not over 45° C., preferably between about 4° C. and about 40° C., more preferably between about 25° C. and about 40° C. for between about 0.5 and 40 hours, preferably between about 1 and about 20 hours. The solvent is not particularly restricted provided that it does not interfere with the reaction, and thus includes, but is not limited to, buffers at between about pH 5 and about pH 9, such as citrate buffer, phosphate buffer, Tris buffer and acetate buffer.

The detection agent is a molecule, optionally labeled with a detectable label as described above. The detection agent is preferably in excess so that essentially all target exogenous GDF-8 modulating agent that may be present in the biological sample will be bound. Detection may be qualitative or quantitative. In some embodiments, the detection agent will comprise a label that will be easily detected by visual means without the aid of instruments. The detection agent may also be detected with instruments. In methods such as surface plasmon resonance, binding of a GDF-8 modulating agent to a captive agent is detected without the addition of a label, for example.

The detection agent is immobilized by specific binding to an exogenous GDF-8 modulating agent, for example. In one embodiment, the detection agent is an anti-human IgG antibody conjugated to horseradish peroxidase. The presence or absence of the exogenous agent in a sample is evaluated by measuring the label activity, which may depend on the kind of label used to measure the detection agent.

In some embodiments, a “direct” label may be any molecule bound or conjugated to a specific binding member which is capable of spontaneously producing a detectable signal without the addition of ancillary reagents. Some examples include a radioisotope (e.g., ¹²⁵I, ³H, ¹⁴C), a heavy metal, a fluorophore (e.g., luciferase, green fluorescent protein, fluorescein isothiocyanate, tetramethylrhodamine isothiocyanate, 1-N-(2,2,6,6-tetramethyl-1-oxyl-4-piperidyl)-5-N-(aspartate)-2,4-dinitrobenzene), a dye (e.g., phycocyanin, phycoerythrin, Texas red, o-phthalaldehyde), luminescent molecules, including chemiluminescent and bioluminescent molecules, colloidal gold particles, colloidal silver particles, other colloidal metal particles, Europium, polystyrene dye particles, minute colored particles such as dye sols, and colored latex particles. Many such substances are well known to those skilled in the art.

In some embodiments, the label is an enzyme such as, e.g., alkaline phosphatase, peroxidase (e.g. horseradish peroxidase), glucose oxidase, or β-galactosidase. The substrates to be used with the specific enzymes are generally chosen for the production, in the presence of the corresponding enzyme, of a detectable change in color, fluorescence, or luminescence. The enzyme is generally conjugated to the detection agent by glutaraldehyde or periodate cross-linking. In certain embodiments the detection agent is a peroxidase-conjugated antibody, such as a monoclonal antibody that specifically binds to the GDF-8 modulating agent or that specifically binds a complex that includes the modulating agent, or to GDF-8 protein. As will be readily recognized, however, a wide variety of different conjugation techniques exist, and are applicable to a variety of detection agents (set forth above), and are readily available to the skilled artisan.

In a preferred embodiment, the enzyme-labeled antibody is added to the GDF-8 modulating agent/capture agent complex, and allowed to bind. The excess reagent is washed away, and a solution containing an appropriate substrate is then added to reaction vessel. The substrate undergoes an enzyme-catalyzed reaction resulting in a spectrophotometrically-measurable change that is indicative of the amount of agent present in the sample.

Peroxidase, when incubated with soluble substrates (e.g., 3,3′,5,5′ tetramethylbenzidine (TMB), o-phenylene diamine (OPD), 2,2′-azino-di [3-ethyl-benzthiazoline] sulfonate (ABTS), para nitrophenyl phosphate, luminol, polyphenols, acridine esters, and luciferin), results in a chromogenic or luminescent change in the substrate that can be detected spectroscopically. Typically, after a fixed incubation period with the substrate, the reaction is quenched (e.g., by acidification), and the result is quantified by measuring optical density (absorbance) or luminescence. Absorbance results can be compared with the OD values in the linear range for chomogenic reactions, and luminescent immunoassays are measured in relative light units (RLU). As a further alternative, any combination of reagents that results in binding and the generation of a practicable dose-response signal may be used (e.g., radiolabelled agents, enzyme/substrate reagents, or detection amplification systems utilizing biotin/avidin, for example).

In yet other embodiments, the label is biotin, a hapten, or an epitope tag (e.g., histidine-tag, HA-tag (hemagglutinin peptide), maltose binding protein, AviTag®, or glutathione-S-transferase), which can be detected by the addition of a labeled detection agent that interacts with the label associated with the GDF-8 modulating agent complex. A biotin-labeled (“biotinylated”) detection agent may be detected through its interaction with an avidin-enzyme, e.g., avidin-horseradish peroxidase, conjugate after sequential incubation with the avidin-enzyme conjugate and a suitable chromogenic or fluorogenic substrate.

In step (d) a GDF-8 modulating agent complex associated with the surface of the reaction vessel is detected by qualitative or quantitative assessment of the signal of the label. The label can be measured directly, e.g., by fluorescence or luminescence, or indirectly, via addition of a substrate. The label can also be measured, following incubation with an additional reagent.

In an embodiment in which the label is biotin, an avidin-conjugated enzyme (which is in some preferred embodiments horseradish peroxidase), is added in a subsequent step. The avidin conjugate binds to the immobilized detection agent. Excess avidin conjugate is washed away. A substrate of the enzyme is then added, resulting in a measurable change in, e.g., color, fluorescence, or luminescence. In some embodiments the substrate of horseradish peroxidase is 3,3′,5,5′-tetramethylbenzidine.

In other embodiments, this method enables the detection in a complex biological sample of a GDF-8 modulating agent that specifically binds with follistatin, various GDF-8 binding receptors, activin, GDF-8 propeptide, or other GDF-8 modulating agents in biological samples. In certain embodiments, the protein that specifically binds to GDF-8 (for example, chosen from the preceding list) is the capture agent, and the capture agent is immobilized on a surface of the reaction vessel. In a preferred embodiment, this method enables the detection of an exogenous GDF-8 modulating agent in a biological sample from an individual, based on competition or interference with an interaction of mature GDF-8 with one or more specific binding partners (see below).

The detection agent in steps (c) and (d) is, insome embodiments, an antibody, such as a mouse anti-human Ig antibody, as described in Example 1. In a preferred embodiment, the method to detect an exogenous GDF-8 modulating agent in a biological sample comprises: (a) contacting a mature GDF-8 protein with a surface of a reaction vessel; (b) adding a biological sample to the reaction vessel; (c) adding a detection agent to the reaction vessel; and (d) detecting a GDF-8 modulating agent/GDF-8 protein complex associated with the surface of the reaction vessel. In a preferred aspect, the mature GDF-8 protein comprises a biotin moiety and contacts the surface via the biotin moiety. In a preferred aspect, the molar ratio of biotin moiety to mature GDF-8 protein is between about 0.5:1 and 4:1. In a further preferred aspect, the GDF-8 modulating agent is MYO-029.

In a further embodiment, a method to detect an exogenous GDF-8 modulating agent in a biological sample is provided, comprising: (a) contacting a capture agent with a surface of a reaction vessel, wherein the capture agent is chosen from a GDF-8 protein and a protein that specifically binds to a GDF-8 protein; (b) adding a biological sample to the reaction vessel; (c) adding a detection agent to the reaction vessel; and (d) detecting a GDF-8 modulating agent/capture agent complex associated with the surface of the reaction vessel, thereby detecting an exogenous GDF-8 modulating agent in the biological sample. In yet another embodiment, a method to detect a GDF-8 modulating agent in a biological sample is provided, the method comprising: (a) contacting a GDF-8 receptor with a surface of a first and a second reaction vessel; (b) adding a biological sample and a GDF-8 protein to the first reaction vessel of (a); (c) adding a control sample and a GDF-8 protein to the second reaction vessel of (a); (d) adding a detectable marker to the first and second reaction vessels; and (e) comparing the detectable marker signal in the first reaction vessel to the signal in the second reaction vessel, thereby detecting the GDF-8 modulating agent in the biological sample.

Competitive ELISA

In further embodiments of the invention, the in vitro immunoassay is a competitive ELISA. In one method provided herein, the immunoassay comprises the steps of: (a) contacting a soluble GDF-8 receptor with a surface of a reaction vessel; (b) adding a biological sample to the reaction vessel; (c) adding a labeled GDF-8 protein to the reaction vessel; and (d) detecting the amount of labeled GDF-8 protein/GDF-8 receptor complex associated with the surface in the presence and absence of the biological sample, wherein a reduction in the amount of labeled GDF-8 protein/GDF-8 receptor complex in the presence of the biological sample detects an exogenous GDF-8 modulating agent in the biological sample. In certain embodiments, the method further comprises the step of incubating the biological sample with the labeled GDF-8 protein prior to adding the sample to the reaction vessel. In additional embodiments, a biotinylated GDF-8 protein, for example as described above, may be used as the detection agent.

GDF-8 Inhibitors

A GDF-8 modulating agent, including a GDF-8 inhibitor, may be detected by the methods provided herein. It may also be used in the methods, for example as a detection agent in a binding assay. GDF-8 inhibitors may interact with GDF-8 itself. Alternatively, inhibitors may interact with a GDF-8 receptor (such as ActRIIB) or other binding partner or they may act indirectly. GDF-8 inhibitors are a subset of GDF-8 modulating agents, and include antibodies (against, e.g., GDF-8 and/or a GDF-8 receptor), soluble receptors, other proteins (including those that bind to GDF-8 and/or a GDF-8 receptor), modified forms of GDF-8 or fragments thereof, propeptides, peptides, and mimetics of all of these inhibitors. Nonproteinaceous inhibitors include, for example, nucleic acids.

It will be understood by one of ordinary skill in the art that certain amino acids in a sequence of any protein may be substituted for other amino acids without adversely affecting the activity of the protein. It is thus contemplated that various changes may be made in the amino acid sequences of the GDF-8 modulating agents and GDF-8 inhibitors of the invention, or DNA sequences encoding the same, without appreciable loss of their biological activity or utility. Such changes may include, but are not limited to, deletions, insertions, truncations, and substitutions.

The primary sequence of an amino acid or nucleotide-based agent or inhibitor may differ from a reference sequence. For example, a nucleotide sequence may associate with a related sequence under “highly stringent” or “high stringency” conditions for hybridization and washing. Such conditions are known to those skilled in the art and can be found in, for example, “Current Protocols in Molecular Biology,” John Wiley & Sons, N.Y. (1989), 6.3.1-6.3.6. Both aqueous and nonaqueous conditions as described in the art can be used. One example of highly stringent hybridization conditions is hybridization in 6× sodium chloride/sodium citrate (SSC) at about 45° C., followed by at least one wash in 0.2×SSC, 0.1% sodium dodecyl sulfate (SDS) at 50° C. Other examples of highly stringent hybridization conditions include hybridization in 6×SSC at about 45° C. (or 50° C., 60° C., or 65° C.) followed by at least one wash in 0.2×SSC, 0.1% SDS at about 55° C., 60° C., or 65° C. Highly stringent conditions may also be hybridization in 0.5M sodium phosphate, 7% SDS at 65° C., followed by at least one wash at 0.2×SSC, 1% SDS at 65° C.

One of skill in the art will recognize that a proteinaceous GDF-8 modulating agent or GDF-8 inhibitor may contain a number of conservative changes to its amino acid sequence without altering its biological properties. Conservative amino acid modifications are based on the relative similarity of the amino acid side-chain substituents, for example, their hydrophobicity, hydrophilicity, charge, size, and the like. Exemplary conservative substitutions are well known to those of skill in the art and include: arginine and lysine; glutamate and aspartate; serine and threonine; glutamine and asparagine; and valine, leucine, and isoleucine. Furthermore, functional fragments of a proteinaceous GDF-8 modulating agent or GDF-8 inhibitor are provided herein. It is expected that such fragments would specifically bind to GDF-8 and/or inhibit a GDF-8 activity. In an embodiment of the invention, a GDF-8 modulating agent, or a functional fragment thereof, specifically binds to mature GDF-8 or a fragment thereof, whether it is in monomeric form, active dimer form, or complexed in a GDF-8 latent complex.

When referring to an amino acid or nucleic acid sequence, the phrase “substantially identical” or “substantially similar” means that the relevant amino acid or nucleotide sequence, such as of the GDF-8 inhibitors of the invention, will be identical to or have insubstantial differences (through conserved amino acid substitutions) in comparison to the sequences which are disclosed. Nucleotide and polypeptides of the invention include, for example, those that are at least about 60%, 65%, 70%, 75%, 80%, 85%, 90%, 92%, 93%, 94%, 95%, 96%, 97%, 98%, or 99% identical in sequence to nucleic acid molecules and polypeptides disclosed.

For polypeptides, at least 20, 30, 50, 100, or more amino acids will be compared between the original polypeptide and the variant polypeptide that is substantially identical to the original. For nucleic acids, at least 50, 100, 150, 300 or more nucleotides will be compared between the original nucleic acid and the variant nucleic acid that is substantially identical to the original. Alternatively, a comparison may be done on at least 60%, 70%, 80%, 90% of the original amino acid or nucleic acid sequence. Thus, a variant could be substantially identical in a region or regions, but divergent in others. Percent identity between two sequences is determined by standard alignment algorithms such as, for example, Basic Local Alignment Tool (BLAST) described in Altschul et al., J. Mol. Biol. 215:403-410 (1990), the algorithm of Needleman et al., J. Mol. Biol. 48:444-453 (1970), or the algorithm of Meyers et al., Comput. Appl. Biosci. 4:11-17 (1988).

The term “variant” refers to nucleotide and amino acid sequences that are substantially identical or similar to the nucleotide and amino acid sequences of GDF-8 inhibitors (as well as GDF-8 itself) provided, respectively. Variants can be naturally occurring, for example, naturally occurring human and non-human nucleotide sequences, or they can be generated artificially. Examples of variants are those resulting from alternative splicing of the mRNA, including both 3′ and 5′ spliced variants, point mutations and other mutations, or proteolytic cleavage of the proteins. Variants include nucleic acid molecules or fragments thereof and amino acid sequences and fragments thereof, that are substantially identical or similar to other nucleic acids (or their complementary strands when they are optimally aligned (with appropriate insertions or deletions) or amino acid sequences respectively. In one embodiment, there is at least about 60%, 65%, 70%, 75%, 80%, 85%, 90%, 92%, 93%, 94%, 95%, 96%, 97%, 98%, or 99% identity between a nucleic acid molecule or protein of the invention and another nucleic acid molecule or protein respectively, when optimally aligned. Alternatively, an entire epitope may be inserted into a non-homologous molecule. Additionally, variants include proteins or polypeptides that exhibit GDF-8 activity or inhibit GDF-8 activity, as discussed in this application.

The GDF-8 inhibitors can be glycosylated, pegylated, or linked to another nonproteinaceous polymer. The GDF-8 inhibitors of the invention may be modified to have an altered glycosylation pattern (i.e., altered from the original or native glycosylation pattern). As used herein, “altered” means having one or more carbohydrate moieties modified, and/or having one or more glycosylation sites changed in the original inhibitor. Addition of glycosylation sites to the GDF-8 inhibitors may be accomplished by altering the amino acid sequence to contain glycosylation site consensus sequences well known in the art. Another means of increasing the number of carbohydrate moieties is by chemical or enzymatic coupling of glycosides to the amino acid residues of the inhibitor. These methods are described in WO 87/05330, and in Aplin et al., Crit. Rev. Biochem. 22:259-306 (1981). Removal of any carbohydrate moieties present on the receptor may be accomplished chemically or enzymatically as described by Hakimuddin et al., Arch. Biochem. Biophys. 259:52 (1987); Edge et al., Anal. Biochem. 118:131(1981); and by Thotakura et al., Meth. Enzymol. 138:350 (1987).

1. Antibodies

Antibodies that inhibit GDF-8 activity are within the scope of the GDF-8 modulating agents provided herein. Antibodies can be made, for example, by traditional hybridoma techniques (Kohler et al., Nature, 256:495-499 (1975)), recombinant DNA methods (U.S. Pat. No. 4,816,567), or phage display techniques using antibody libraries (Clackson et al. Nature, 352:624-628 (1991); Marks et al., J. Mol. Biol., 222:581-597 (1991)). For various other antibody production techniques, see, e.g., Antibodies: A Laboratory Manual, (Harlow et al., eds., Cold Spring Harbor Laboratory 1988); and Antibody Engineering, 2nd ed., (Borrebaeck, ed., Oxford University Press 1995). Antibodies may be fully human or humanized. In certain embodiments, antibodies may have an altered or mutated Fc region as described in subsequent sections.

The affinity of antibodies according to this invention may be between 10⁶ M⁻¹ and 10¹¹ M⁻¹, and may be between 10⁸ M⁻¹ and 10¹⁰ M⁻¹, for example. The antibodies of the invention may inhibit GDF-8 activity in vitro or in vivo. The disclosed antibodies may inhibit GDF-8 activity associated with negative regulation of skeletal muscle mass and bone density and/or may affect clearance or bioavailability of GDF-8.

2. Antibodies Against GDF-8

Antibodies that are GDF-8 modulating agents may bind to the GDF-8 protein itself. In particular embodiments, the antibodies specifically bind to a GDF-8 protein or GDF-8/GDF-8 receptor complex. Such antibodies may be capable of binding mature GDF-8 with high affinity, and may bind the mature protein whether it is in monomeric form, active dimer form, or complexed in a GDF-8 latent complex. In preferred embodiments, the antibodies that bind to GDF-8 protein are neutralizing antibodies. In certain embodiments, GDF-8 antibodies block the binding of GDF-8 to its receptor, for example as measured in a competitive binding assay. Antibodies to GDF-8 sequences are discussed in U.S. Pat. Nos. 5,827,733 and 6,096,506, for example.

A. MYO-029, MYO-028, and MYO-022

The MYO-029, MYO-028, and MYO-022 antibodies can be used in the methods of the invention, and these antibodies are described in further detail in U.S. Patent Pub. No. 2004/0142382-A1, relevant portions of which are herein incorporated by reference including sequence, structure, fragment, binding, biological activity, and antigen epitope information for the antibodies, for example. For example, characteristics of certain neutralizing antibodies, including MYO-029, are described in U.S. Patent Pub. No. 2004/0142382-A1 in paragraphs 54-90, and claims 1-42. These antibodies are capable of binding mature GDF-8 with high affinity, inhibiting GDF-8 activity in vitro and in vivo as demonstrated, for example, by inhibition of ActRIIB binding and reporter gene assays, and inhibiting GDF-8 activity associated with negative regulation of skeletal muscle mass and bone density.

DNA and amino acid (M) sequences of MYO-029, MYO-028, and MYO-022 antibodies, their scFv fragments, V_{H} and V_{L} domains, and CDRs are set forth in the Sequences Listing (MYO-029) and description of U.S. Patent Pub. No. 2004/0142382-A1 (MYO-029, MYO-028, and MYO-022). The sequences of heavy and light chains excluding the V_{H} and V_{L} domains are identical in MYO-029, MYO-028, and MYO-022. In one preferred embodiment, sequences of MYO-029 are set forth as SEQ ID NOs:3-20.

B. JA-16

The JA-16 antibody binds to a mature GDF-8 protein as set forth in SEQ ID NO:1, and is described in further detail in L-A Whittemore et al., Biochem. and Biophys. Res. Commun. 300:965-971 (2003), as well as in U.S. Patent Pub. No. 2003/0138422-A1, relevant portions of each (including sequence, structure, fragment, binding, biological activity, and antigen epitope, for example), is herein incorporated by reference. In particular, antibody inhibitors of U.S. Patent Pub. No. 2003/0138422-A1, are described in paragraphs 56-70, 93-110, and claims 1-54, for example.

3. Antibodies Against a GDF-8 Receptor

Antibodies that bind to a GDF-8 receptor are within the scope of the GDF-8 modulating agents detected with the methods of this invention. These antibodies may affect the binding of GDF-8 to its receptor, or they block the activity of the receptor after binding of GDF-8. Antibodies can be developed against the whole receptor protein, or against only the extracellular domain. Antibodies may be developed against ActRIIB, ActRIIB variants, and other receptors for GDF-8 (see, e.g., U.S. Patent Pub. No. 2004/0223966-A1; U.S. Patent Pub. No. 2004/0077053-A1; WO 00/43781).

4. Modified Soluble Receptors

Modified soluble receptors of GDF-8, which are themselves GDF-8 modulating agents, may be used in the invention to detect other GDF-8 modulating agents. Soluble receptors may comprise all or part of the extracellular domain of a GDF-8 receptor, such as ActRIIB or ActRIIA which bind to GDF-8 in assays well known in the art (Lee et al., Proc. Natl. Acad. Sci. U.S.A. 98:9306-9311 (2001)). Activin type II receptors are highly conserved, and recombinant soluble forms of the same are provided in Attisano et al., Mol. and Cell Biol. 16:1066 (1996); Woodruff, Pharmacology 55:953 (1998); and R.& D Systems Cat. No. 339-R (a human Act RIIB-Fc chimera), for example. GDF-8 receptor structural and functional properties, as well as assays for the activity the same are provided, for example in U.S. Pat. Nos. 6,656,475 and 6,696,260, and U.S. Patent Pub. No. 2004/0077053-A1. Further, activin receptors, including activin type II receptors, are provided, for example in U.S. Pat. No. 6,835,544, describing the extracellular ligand-binding domains of the same.

Such receptors may be produced recombinantly or by chemical or enzymatic cleavage of the intact receptor. The modified soluble receptors of the invention reduce the ability of GDF-8 to activate native GDF-8 receptor in the body and inhibit GDF-8 activity. The sequences for the ActRIIB receptor, including description of the extracellular domain, specific fragments and variants of the receptor are set forth in U.S. Pat. No. 6,656,475, for example.

A. Receptor Fusions

The modified soluble receptors of the invention may be made more stable by fusion to another protein or portion of another protein. Increased stability is advantageous for therapeutics to allow administration of a lower dose or at less frequent intervals. Fusion to at least a portion of an immunoglobulin, such as the constant region of an antibody, optionally an Fc fragment of an immunoglobulin, can increase the stability of a modified soluble receptor or other proteins of the invention. (See, e.g., Spiekermann et al., J. Exp. Med. 96:303-310 (2002)).

B. ActRIIB Fc Fusions

ActRIIB Fc fusion inhibitors, described in further detail in U.S. Patent Pub. No. 2004/0223966-A1, relevant portions of which are herein incorporated by reference, comprise a modified activin type 11 receptor ActRIIB that binds GDF-8 and inhibits its activity in vitro and in vivo. In particular, the ActRIIB fusion polypeptides inhibit the GDF-8 activity associated with negative regulation of skeletal muscle mass and bone density. The ActRIIB fusion polypeptides of the methods provided herein are soluble and possess pharmacokinetic properties that make them suitable for therapeutic use, e.g., extended circulatory half-life and/or improved protection from proteolytic degradation.

ActRIIB fusion polypeptides may be used, for example, in the methods of the invention to detect GDF-8 modulating agents. These polypeptides comprise a first amino acid sequence derived from the extracellular domain of ActRIIB and stabilizing portion or second amino acid sequence. The first amino acid sequence is derived from all or a portion of the ActRIIB extracellular domain and is capable of binding GDF-8 specifically. In some embodiments, such a portion of the ActRIIB extracellular domain may also specifically bind BMP-11 and/or activin, or other growth factors. In certain embodiments, the ActRIIB is a fragment or truncation of the intact receptor, so long as the shortened sequence is capable of specifically binding GDF-8.

The stabilizing portion, may be an amino acid sequence derived from the constant region of an antibody, particularly the Fc portion, or a mutation of such a sequence. In some embodiments, the amino acid sequence is derived from the Fc portion of an IgG. In related embodiments, the Fc portion is derived from IgG that is IgG1, IgG4, or another IgG isotype. In a particular embodiment, the second amino acid sequence comprises the Fc portion of human IgG1, wherein the Fc portion of human IgG1 has been modified to minimize the effector function of the Fc portion. Such modifications include changing specific amino acid residues which might alter an effector function such as Fc receptor binding (Lund et al., J. Immun., 147:2657-2662 (1991); and Morgan et al., Immunology, 86:319-324 (1995)), or changing the species from which the constant region is derived. Antibodies may have mutations in the C_{H}2 region of the heavy chain that reduce effector function, i.e., Fc receptor binding and complement activation. For example, antibodies may have mutations such as those described in U.S. Pat. Nos. 5,624,821 and 5,648,260. In the IgG1 or IgG2 heavy chain, for example, such mutations may be made at amino acid residues corresponding to amino acids 234 and 237 in the full-length sequence of IgG1 or IgG2. Antibodies may also have mutations that stabilize the disulfide bond between the two heavy chains of an immunoglobulin, such as mutations in the hinge region of IgG4, as disclosed in Angal et al., Mol. Immunol. 30:105-108 (1993).

In certain embodiments, the stabilizing portion is linked to the C-terminus or the N-terminus of the receptor sequence, with or without being linked by a linker sequence. The exact length and sequence of the linker and its orientation relative to the linked sequences may vary. The linker may comprise 2, 10, 20, 30, or more amino acids and is selected based on properties desired such as solubility, length and steric separation, immunogenicity, etc. In certain embodiments, the linker may comprise a sequence of a proteolytic cleavage site, such as the enterokinase cleavage site or other functional sequences useful, for example, for purification, detection, or modification of the fusion protein. One skilled in the art would readily apply such technology to other proteinaceous GDF-8 modulating agents as described herein, creating various fusion proteins.

5. Other Proteins

Other proteins that inhibit GDF-8 activity may be detected in the methods provided herein. Such proteins can interact with GDF-8 itself, inhibiting its activity or binding to its receptor. Alternatively, inhibitors can interact with a GDF-8 receptor (such as ActRIIB) and may be effective in the detection methods of the invention if they block the binding of GDF-8 to its receptor or if they block the activity of the receptor after binding of GDF-8. Inhibitors, of course, may interact with both GDF-8 and its receptor. Inhibitors may also affect GDF-8 activity in other ways, such as by inhibiting the metalloprotease that cleaves an inhibitory GDF-8 propeptide to inactivate it (see, e.g., U.S. Patent Pub. No. 2004/0138118-A1).

A. Proteins that Specifically Bind to GDF-8

Proteins that bind to GDF-8 and inhibit its activity or affect its clearance are acceptable for use in the methods of the invention. While some proteins are known, additional proteins can be isolated using the various assays such as the ActRIIB binding assay, immunoassays, or reporter gene assays described herein. Samples of proteins may be screened, as well as libraries of proteins.

B. GDF-8 Propeptide

GDF-8 propeptide can be used as an inhibitor of GDF-8. Because naturally occurring GDF-8 propeptides have a short in vivo half-life thereby reducing their effectiveness as pharmacological inhibitors of GDF-8 activity, a GDF-8 propeptide inhibitor includes modified and stabilized forms of GDF-8 propeptides having improved pharmacokinetic properties, specifically an increased circulatory half-life. See U.S. Patent Pub. No. 2003/0104406-A1, relevant portions of which are herein incorporated by reference.

Such modified GDF propeptides include fusion proteins comprising a GDF propeptide and an Fc region of an IgG molecule (as a stabilizing protein). These GDF inhibitors may comprise a GDF propeptide (for example as set forth in SEQ ID NO:5 or 11) or a fragment or variant of said propeptide which retains one or more biological activities of a GDF propeptide. GDF-8 propeptides used in the methods of the invention may be synthetically produced, derived from naturally occurring (native) GDF-8 propeptides, or be produced recombinantly, using any of a variety of reagents, host cells and methods which are well known in the art of genetic engineering. In one embodiment, the modified GDF-8 propeptide comprises a human GDF-8 propeptide covalently linked to an IgG molecule or a fragment thereof. The GDF-8 propeptide may be linked directly to the Fc region of the IgG molecule, or linked to the Fc region of the IgG molecule via a linker peptide. Further proteins that bind to GDF-8, including propeptides of GDF-8 are provided in WO 00/43781.

C. Follistatin and Follistatin-Domain Containing Proteins

Proteins comprising at least one follistatin domain modulate the level or activity of growth and differentiation factor-8 (GDF-8), and may be used for treating disorders that are related to the modulation of the level or activity of GDF-8. Both follistatin itself and follistatin domain containing proteins (described in U.S. Patent Pub. Nos. 2003/0162714-A1 and 2003/0180306-A1), relevant portions of both of which are herein incorporated by reference) may be used in the invention (see also, Lee et al., Proc. Natl. Acad. Sci U.S.A. 98:9306-9311 (2001)). Administration of these proteins to a human or an animal may be detected using the methods of the invention.

Proteins containing at least one follistatin domain will bind and inhibit GDF-8. Examples of proteins having at least one follistatin domain include, but are not limited to follistatin, follistatin-like related gene (FLRG), FRP (flik, tsc 36), agrins, osteonectin (SPARC, BM40), hevin (SC1, mast9, QR1), IGFBP7 (mac25), and U19878. GASP1 and GASP2 are other examples of proteins comprising at least one follistatin domain.

A follistatin domain, as stated above, is defined as an amino acid domain or a nucleotide domain encoding for an amino acid domain, characterized by cysteine rich repeats. A follistatin domain typically encompasses a 65-90 amino acid span and contains 10 conserved cysteine residues and a region similar to Kazal serine protease inhibitor domains. In general, the loop regions between the cysteine residues exhibit sequence variability in follistatin domains, but some conservation is evident. The loop between the fourth and fifth cysteines is usually small, containing only 1 or 2 amino acids. The amino acids in the loop between the seventh and eighth cysteines are generally the most highly conserved containing a consensus sequence of (G,A)-(S,N)-(S,N,T)-(D,N)-(G,N) followed by a (T,S)-Y motif. The region between the ninth and tenth cysteines generally contains a motif containing two hydrophobic residues (specifically V, I, or L) separated by another amino acid.

A follistatin domain-containing protein will comprise at least one, but possibly more than one, follistatin domain. The term also refers to any variants of such proteins (including fragments; proteins with substitution, addition or deletion mutations; and fusion proteins) that maintain the known biological activities associated with the native proteins, especially those pertaining to GDF-8 binding activity, including sequences that have been modified with conservative or non-conservative changes to the amino acid sequence. These proteins may be derived from any source, natural or synthetic. The protein may be human or derived from animal sources, including, but not limited to, bovine, chicken, murine, rat, porcine, ovine, turkey, baboon, and fish.

Proteins comprising at least one follistatin domain, which may bind GDF-8, may be isolated using a variety of methods. For example, one may use affinity purification using GDF-8. In addition, one may use a low stringency screening of a cDNA library, or use degenerate PCR techniques using a probe directed toward a follistatin domain. As more genomic data becomes available, similarity searching using a number of sequence profiling and analysis programs, such as MotifSearch (Genetics Computer Group, Madison, Wis.), ProfileSearch (GCG), and BLAST (NCBI) could be used to find novel proteins containing significant homology with known follistatin domains.

D. Proteins Binding to GDF-8 Receptor

Proteins that bind to a GDF-8 receptor (such as ActRIIB) and inhibit the binding of GDF-8 to the receptor or the activity of the receptor itself are acceptable for use in the methods of the invention for detecting GDF-8 modulating agents. Such proteins can be isolated using screening techniques and the ActRIIB binding assay or reporter gene assays described herein. Samples of proteins may be screened, as well as libraries of proteins.

E. Fusions with any of the Proteins Binding to GDF-8 or GDF-8 Receptor

Fusion proteins of any of the proteins that bind to GDF-8 or a GDF-8 receptor can be made more stable by fusion to another protein or portion of another protein. Modification of a GDF-8 modulating agent to increase stability is advantageous for therapeutics as they can be administered at a lower dose or at less frequent intervals. Fusion to at least a portion of an immunoglobulin, such as the constant region, optionally an Fc fragment of an immunoglobulin, can increase the stability of these proteins. The preparation of such fusion proteins is well known in the art and can be performed easily. (See, e.g., Gerburg Spiekermann (2002) J. Exp. Med., 96:303-310).

A GDF-8 propeptide Fc fusion inhibitor, described in greater detail in U.S. Patent Pub. No. 2003/0104406-A1, relevant portions of which are hereby incorporated by reference, comprises a polypeptide cleaved from the amino-terminal domain of the GDF-8 precursor protein, covalently linked with the Fc region of an IgG molecule or fragment thereof.

The GDF-8 propeptide Fc fusion inhibitor comprises a human GDF-8 propeptide or a mutant of GDF-8 propeptide, and the Fc region of an IgG1, an IgG4, or an IgG1 modified for reduced effector function. The GDF-8 propeptide may be modified to include stabilizing modifications.

F. Inhibitors of Protease Activation of the GDF-8 Latent Complex

Inhibitors of protease activation of the GDF-8 latent complex are described in U.S. Patent Pub. No. 2004/0138118 A1, relevant portions of which are incorporated herein by reference. Certain proteases cleave the propeptide, either in a free form or when it is associated with a mature GDF-8 dimer, rendering it unable to bind to and inhibit the activity of the mature GDF-8 dimer. As such, the proteases can convert a small latent complex (mature GDF-8 associated with and inhibited by propeptide) into active GDF-8. Once the propeptide has been cleaved it cannot bind to and inactivate the mature GDF-8 dimer. Inhibitors of protease activation of the GDF-8 small latent complex will enhance propeptide binding to mature GDF-8 dimers and inhibit GDF-8 activity. These inhibitors may competitively bind the protease, preventing it from binding the native latent complex, or they may also bind the mature GDF-8 dimer creating an inactive inhibitor-mature dimer complex.

Metalloproteases are exemplified by the BMP-1/TLD family of metalloproteases, which includes at least four mammalian proteins, BMP-1 (Wozney et al., Science 242:1528-1534, 1988), mammalian Tolloid (mTLD; Takahara et al., J. Biol. Chem. 269:32572-32578, 1994), mammalian Tolloid-like-1 (mTLL-1; Takahara et al., Genomics 34:157-165, 1996), and mammalian Tolloid-like-2 (mTLL-2; Scott et al., Devel. Biol. 213:283-300, 1999), each of which are incorporated herein by reference.

Various metalloprotease inhibitor GDF-8 modulating agents, are described in U.S. Patent Pub. No. 2004/0138118 A1, including antibody, nucleic acid and peptide based agents, and are incorporated herein by reference. Inhibitors of protease activation of the GDF-8 small latent such as agents that inhibit metalloprotease activity can include any type of molecule, including, for example, a peptide, peptide derivative such as a peptide hydroxamate or a phosphinic peptide, or peptoid and can be identified through the screening assays of U.S. Patent Pub. No. 2004/0138118 A1 (see also, U.S. Patent Pub. No. 2005/0043232 A1).

Particular agents that inhibit protease activation of the GDF-8 small latent complex include peptides that compete for the metalloprotease enzyme with the propeptide GDF-8. These peptides can comprise a portion of the propeptide, a portion of the full length GDF-8 polypeptide containing the propeptide portion, or a derivative of a GDF-8 polypeptide having a mutation of a cleavage site for the metalloprotease. As described in the above U.S. patent publications, in one embodiment, a derivative of a peptide portion of GDF-8 is a peptide that corresponds to a GDF-8 propeptide. In one aspect of this embodiment, the derivative is a propeptide having a mutation of the metalloprotease cleavage site, for example, a substitution, deletion, or insertion of an amino acid at or in sufficient proximity to the cleavage site such that the metalloprotease has altered cleavage activity with respect to the peptide agent. Derivative or modified peptides can have improved stability to a protease, an oxidizing agent or other reactive matenal that the peptide may encounter in a biological environment, and may include, for example the modifications described above.

Inhibitory antibodies against the metalloprotease enzymes, as well as antibodies that specifically bind to such peptide and antibody-based GDF-8 modulating agents, can also be used in this invention and can easily be generated by known techniques in the art.

Peptide agents may be approximately 10, 20, 30, 40, or 50 amino acid residues or more in length, containing wild type or mutant GDF-8 propeptide sequences, or derivatives thereof. For example, peptides having one or more amino acid changes at the P1 position (just upstream of the metalloprotease cleavage site) or the P1′ position (just downstream of the metalloprotease cleavage site) may be changed. In certain GDF-8 modulating agents, an aspartic acid to alanine substitution at the P1′ position (corresponding to position 76 of SEQ ID NO:2) is included in a peptide that is 10, 20, 30, 40 and 50 amino acids in length related to wild type GDF-8 propeptide sequence (U.S. Patent Pub. No. 2004/0138118 A1).

Such GDF-8 modulating agents may be detected and/or identified, for example, in a reporter gene assay, GDF-8 capture, or competitive binding ELISA, as described herein. Exemplary detection agents that will detect such GDF-8 modulating agents that modulate metalloprotease activation of the GDF-8 latent complex include, but are not limited to, antibodies to the agents, mature GDF-8 protein, or portions thereof that bind to a propeptide-based agent, and metalloprotease sequences comprising the substrate binding portion of one or more metalloproteases of the BMP-1/TLD family of metalloproteases, such as could be used in a competition assay.

6. Mimetics of GDF-8 Inhibitors

Mimetics of the GDF-8 inhibitors used in the methods of the invention may also be detected by the methods described herein. Any synthetic analogue of these GDF-8 inhibitors, especially those with improved in vitro characteristics such as having a longer half-life, or being less easily degraded by the digestive system, are useful.

Mimetics of antibodies against GDF-8, antibodies against GDF-8 receptor, modified soluble receptors and receptor fusions, and other proteins binding to GDF-8 such as GDF-8 propeptide, mutated GDF-8 propeptide, follistatin and follistatin-domain containing proteins, and Fc fusions thereof may all be used in the invention.

These mimetics will be effective in the invention if they block the activity of GDF-8, namely if they block the binding of GDF-8 to its receptor. Mimetics that are most effective in this invention will have the property of binding specifically to GDF-8 or the GDF-8/GDF-8 receptor complex. Such mimetics may be capable of binding mature GDF-8 with high affinity, and may bind the mature protein whether it is in monomeric form, active dimer form, or complexed in a GDF-8 latent complex. The mimetics of the invention may inhibit GDF-8 activity in vitro and in vivo as demonstrated, for example, by inhibition of ActRIIB binding and reporter gene assays. Further, the disclosed mimetics may inhibit GDF-8 activity associated with negative regulation of skeletal muscle mass and bone density.

7. Nonproteinaceous Inhibitors

Nonproteinaceous inhibitors include, for example, nucleic acids.

A. Nucleic Acids

The terms “polynucleotide,” “oligonucleotide,” and “nucleic acid” refer to deoxyribonucleic acid (DNA) and, where appropriate, to ribonucleic acid (RNA), or peptide nucleic acid (PNA). The term should also be understood to include nucleotide analogs, and single or double stranded polynucleotides (e.g., siRNA). Examples of polynucleotides include but are not limited to plasmid DNA or fragments thereof, viral DNA or RNA, antisense RNA, etc. The term “plasmid DNA” refers to double stranded DNA that is circular. “Antisense,” as used herein, refers to a nucleic acid capable of hybridizing to a portion of a coding and/or noncoding region of mRNA by virtue of sequence complementarity, thereby interfering with translation from the mRNA. The terms “siRNA” and “RNAi” refer to a nucleic acid which is a double stranded RNA that has the ability to induce degradation of mRNA thereby “silencing” gene expression. Typically, siRNA is at least 15-50 nucleotides long, e.g., 20, 21, 22, 23, 24, 25, 26, 27, 28, 29, or 30 nucleotides in length.

Nucleic acids that that can block an activity of GDF-8 may be detected, for example, by methods provided herein. Such inhibitors may encode proteins that interact with GDF-8 itself. Alternatively, such inhibitors may encode proteins that can interact with a GDF-8 protein or GDF-8 receptor (such as ActRIIB) and may express GDF-8 inhibitors of the invention. Alternatively, antisense nucleic acids may be used to inhibit the production of GDF-8 or a receptor of GDF-8 (such as ActRIIB). Antisense sequences can interact with complementary coding sequences to upset function, which may serve to inhibit GDF-8 or GDF-8 receptor production.

The nucleic acids for use in the invention are identified, for example, using the ActRIIB binding assay and reporter gene assays described above. Detection agents for nucleotide based GDF-8 modulating agents will include, for example, complementary nucleotides or antibodies that specifically bind to the agent.

While the disclosure of the present invention refers to preferred embodiments for detecting GDF-8 modulating agents capable of binding to a GDF-8 protein, it is recognized that GDF-8 modulating agents that modulate other GDF-8 activities may be detected using the methods of the present invention. Similarly, although the disclosure of the present invention is directed to detecting and/or monitoring GDF-8 modulating agent levels in humans and other mammals in connection with in vivo administration of diagnostic or therapeutic products, it will be recognized that the methodology may be adapted for use in other applications and species as well.

The following examples provide illustrative embodiments of the invention. One of ordinary skill in the art will recognize the numerous modifications and variations that may be performed without altering the spirit or scope of the present invention. Such modifications and variations are encompassed within the scope of the invention. The Examples do not in any way limit the invention.

### EXAMPLES

### Example 1

To detect MYO-029 in a human serum sample, an ELISA was performed as follows. Streptavidin was adsorbed by first adding streptavidin coating solution (100 μL/well) (5 μg/mL ImmunoPure streptavidin (Pierce) in 0.1 M Carbonate/Bicarbonate buffer, pH 9.6) to the wells of a 96 well plate (high binding flat-bottom microtiter) (Costar, Cat. No. 3590). The plate was covered with sealing film and incubated at 2-8° C. overnight. Using an automatic plate washer, the plate was washed four times (4×) with THST buffer (300 μL/well) (50 mM Tris-HCl, pH 8.0, containing 1.0 mM glycine, 0.5 M NaCl, and 0.05% v/v/ Tween 20® (J. T. Baker)), reversing the orientation of the plate after the second wash. To block, 200 μL of blocking buffer (1% bovine albumin (Sigma), 0.02% sodium azide in PBS (Dulbeccos)) was added to each well. The plate was covered with sealing film and incubated for 1-2 hours at room temperature and then washed as above. Biotinylated GDF-8 solution (biotin:GDF-8 molar ratio between 0:1 and 3:1) (100 μL/well) (0.5 μg/mL in THST buffer) was added to each of the plate wells. The plate was sealed and incubated at room temperature with shaking for 2 hours+/−15 minutes.

Calibration standards of MYO-029 were prepared at 90.0, 60.0, 40.0, 26.7, 17.8, 11.9, 7.90, 5.27, and 3.51 ng/mL in THST buffer. A MYO-029 working calibrator solution was prepared of 1080 ng/mL MYO-029 in normal human serum (Bioreclamation, Inc.) The 1080 ng/mL stock was fist diluted 8-fold in assay buffer (THST buffer+4% nonfat dry milk), and then a series of 1.5 fold dilutions of the resulting 135 ng/mL standard were prepared in THST+4% nonfat dry milk+12.5% normal human serum to yield the calibration standard concentrations. MYO-029 calibration standards prepared covering the range from 3.51 to 135 ng/mL are equivalent to 28.1 to 1080 ng/mL in 100% human serum. For human serum, the minimum dilution determined was 1:8. Quality control standards of MYO-029 were separately prepared in duplicate at 135, 270, and 540 ng/mL in THST+4% nonfat dry milk+12.5% normal human serum.

Test samples were diluted 8-fold with THST buffer+4% nonfat dry milk (40 μL of sample with 280 μL buffer). Dilutions higher than 8-fold, were first diluted 1:8 in THST buffer+4% nonfat dry milk and then further diluted in THST buffer+4% nonfat dry milk+12.5% human serum).

The plate with immobilized biotinylated GDF-8 was washed four times (4×) with THST buffer (300 μL/well), reversing the plate after the second wash. The calibration standards (above) were added (100 μL/well) to duplicate to wells in the plate, including duplicate blanks of THST buffer+4% nonfat dry milk+12.5% normal human serum (100 μL/well), and duplicate quality control samples (100 μL/well). Test samples (100 μL/well) were added in duplicate to remaining plate wells.

The plate was covered with sealing film and incubated on a plate shaker for 2 hours+/−10 minutes at room temperature. To remove unbound protein, the plate was washed four times (4×) with THST buffer (300 μL/well), reversing the plate after the second wash.

Mouse anti-human IgG-HRP solution (100 μL/well) (Southern Biotechnology Associates, Inc.) was added at a working dilution determined for each batch. For example, a 1:60,000 dilution of this detection agent in THST was optimal for one lot of anti-human IgG-HRP. The plate was incubated on a plate shaker at room temperature for 1 hour+/−10 minutes and then washed four times (4×) with THST buffer (300 μL/well), reversing the plate after the second wash.

To detect immobilized detection agent, 3,3′,5,5′-tetramethylbenzidine (TMB) peroxidase substrate solution (100 μL/well) (BioFX Laboratories) was added to each of the plate wells. The plate was incubated in the dark at room temperature for approximately 9-12 minutes, and then 0.18M sulfuric acid (100 μL/well) was added to each of the plate wells in the same order as the substrate addition. Optical density was read at wavelength of 450 nm.

Quality control and test sample concentrations were determined by interpolation from the standard curve that is fit with a 4-parameter logistic function using the Drug Metabolism Laboratory Information Management System (Watson), version 7.0.1. Sample concentrations are determined using the following function:

$$y = \frac{a - d}{1 + {( x / c )}^{b}} + d$$

where y=signal (OD); x=concentration; a=signal at zero concentration; d=signal at infinite concentration; c=concentration resulting in signal at approximately midpoint between a and d; b=slope at or around c. Exemplary data of quality control and calibration titration are provided in Table 1. In these data sample concentrations were calculated with: y=1.827 for mean of high Q1,2; x=67.8186; a=0.101805; d=3.74133; c=72.8445; b=1.45512.

Dilution factors were entered to determine final concentration in the test samples. The variability (CV) of the calibration standards was less than or equal to 7.5% over the range of 7.90-90.0 ng/mL in 12.5% human serum, showing quantitative analysis of exogenous MYO-029 levels between about 720 and 60 ng/mL in 100% human serum.

For non-human serum samples, including mouse, rat, monkey, and rabbit serum samples, the assay was performed with minor modifications. These data demonstrated the sensitivity and specificity of the immunoassay for MYO-029 in multiple background matrices. Serviceable serum dilution levels for human, mouse, rat, monkey and rabbit were determined to be at least 1:8, 1:4, 1:4, 1:8, and 1:4, respectively.

TABLE 1

| | Mean | | Back- | | | |
|---|---|---|---|---|---|---|
| | Instrument | | calc'd | Assay | | |
| Sample | Response | Individual | Conc. | Conc. | Nominal | sample |
| Name | (OD 450 nm) | Response | (ng/mL) | (ng/mL) | Conc. | dilution |
| 04_1117 High-QC 1 | 1.827 | 1.845 | 542.549 | 67.8186 | 540 | 1:8 |
| 04_1117 High-QC 2 | | 1.809 | | | 540 | |
| 04_1117 High-QC 3 | 1.749 | 1.798 | 511.341 | 63.9177 | 540 | 1:8 |
| 04_1117 High-QC 4 | | 1.700 | | | 540 | |
| 04_1117 Mid-QC 1 | 0.949 | 0.960 | 256.752 | 32.0941 | 270 | 1:8 |
| 04_1117 Mid-QC 2 | | 0.938 | | | 270 | |
| 04_1117 Mid-QC 3 | 0.983 | 0.933 | 265.883 | 33.2354 | 270 | 1:8 |
| 04_1117 Mid-QC 4 | | 1.032 | | | 270 | |
| 04_1117 Low-QC 1 | 0.493 | 0.488 | 136.063 | 17.0079 | 135 | 1:8 |
| 04_1117 Low-QC 2 | | 0.498 | | | 135 | |
| 04_1117 Low-QC 3 | 0.433 | 0.458 | 119.836 | 14.9794 | 135 | 1:8 |
| 04_1117 Low-QC 4 | | 0.408 | | | 135 | |
| 04_1117 Std 11 | 2.671 | 2.619 | 132.904 | 132.904 | 135 | |
| 04_1117 Std 12 | | 2.722 | | | 135 | |
| 04_1117 Std 21 | 2.243 | 2.206 | 93.064 | 93.064 | 90 | |
| 04_1117 Std 22 | | 2.279 | | | 90 | |
| 04_1117 Std 31 | 1.642 | 1.679 | 58.8564 | 58.8564 | 60 | |
| 04_1117 Std 32 | | 1.604 | | | 60 | |
| 04_1117 Std 41 | 1.159 | 1.133 | 39.4142 | 39.4142 | 40 | |
| 04_1117 Std 42 | | 1.184 | | | 40 | |
| 04_1117 Std 51 | 0.792 | 0.780 | 26.8324 | 26.8324 | 26.7 | |
| 04_1117 Std 52 | | 0.803 | | | 26.7 | |
| 04_1117 Std 61 | 0.529 | 0.526 | 18.2076 | 18.2076 | 17.8 | |
| 04_1117 Std 62 | | 0.532 | | | 17.8 | |
| 04_1117 Std 71 | 0.355 | 0.349 | 12.2393 | 12.2393 | 11.9 | |
| 04_1117 Std 72 | | 0.360 | | | 11.9 | |
| 04_1117 Std 81 | 0.250 | 0.240 | 8.3064 | 8.3064 | 7.9 | |
| 04_1117 Std 82 | | 0.260 | | | 7.9 | |
| 04_1117 Std 91 | 0.167 | 0.166 | 4.64859 | 4.64859 | 5.27 | |
| 04_1117 Std 92 | | 0.168 | | | 5.27 | |
| 04_1117 Std 101 | 0.138 | 0.136 | 3.05552 | 3.05552 | 3.51 | |
| 04_1117 Std 102 | | 0.139 | | | 3.51 | |

### Example 2

Dilutional Linearity: The dilutional linearity of the method was evaluated by analyzing a MYO-029 spiked human serum sample at 11 different dilutions. The 54000 ng/mL sample was initially diluted 1:8 in THST buffer+4% nonfat dry milk followed by a series of dilutions (1:2) in THST buffer+4% nonfat dry milk+12.5% human serum. The dilutions were intended to fall above, within, and below the assay range. The biases for dilutions were determined, with the biases of the samples that fall within the quantitative range of the assay ranging from −9.7% to −0.4%. A trend in the biases was not observed. The observed concentrations decreased as expected, and there was no evidence of a prozone effect.

Specificity: potential non-specific interference from sample matrix (or matrix effect) was investigated by a spiking/recovery experiment using 10 different human serum lots (individual donors) at the MYO-029 spiked concentrations of 0, 135, and 540 ng/mL. Interference from endogenous myostatin (GDF-8) was evaluated by spiking GDF-8 at 0, 1, 2, 10, and 1000 ng/mL into validation samples containing MYO-029 (132, 265, and 529 ng/mL). Endogenous GDF-8 levels are thought to be less than 1 ng/mL. The results for the individual serum samples with and without spiked MYO-029 are displayed in Table 2. At the spiked concentrations of 540 ng/mL, 9 of the 10 sera had mean observed concentrations within 20% of the expected concentration. Upon reanalysis of sera #1, the value was within 15% of the expected concentration. At the spiked concentrations of 135 ng/mL, 8 of the 10 sera had mean observed concentrations within 20% of the expected concentration. Upon reanalysis of sera #3, the value was within 15% of the expected concentration. The high percent bias obtained for sera #1 was confirmed upon reanalysis where the value was still greater than 20% of the expected concentration. Without the addition of MYO-029, all 10 sera had observed concentrations of MYO-029 less than the lower limit of quantitation (i.e., less than 63.2 ng/mL in 100% human serum). The data indicate a lack of a significant matrix effect.

TABLE 2
Matrix Effect for the Quantitation of MYO-029 in 100% Human
Serum

| | | | Measured | |
|---|---|---|---|---|
| | Added | | (Duplicate) | |
| Run #/ | Concentration | | Concentration | |
| Plate ID | (ng/mL) | Serum # | (ng/mL) | Bias |
| 14-060304-sl1 | 135 | 1 | <63.2 | NA |
| 14-060304-sl1 | 540 | 1 | 286 | −47.0% |
| 16-060704-od1 | 135-Repeat | 1 | 91.3 | −32.4% |
| 16-060704-od1 | 540-Repeat | 1 | 461 | −14.7% |
| 14-060304-sl1 | 135 | 2 | 135 | 0.0% |
| 14-060304-sl1 | 540 | 2 | 504 | −6.7% |
| 14-060304-sl1 | 135 | 3 | 164 | 21.5% |
| 16-060704-od1 | 135-Repeat | 3 | 155 | 14.7% |
| 14-060304-sl1 | 540 | 3 | 607 | 12.4% |
| 14-060304-sl1 | 135 | 4 | 159 | 17.8% |
| 14-060304-sl1 | 540 | 4 | 530 | −1.9% |
| 14-060304-sl1 | 135 | 5 | 139 | 3.0% |
| | 540 | 5 | 501 | −7.2% |
| 14-060304-sl1 | 135 | 6 | 124 | −8.1% |
| 14-060304-sl1 | 540 | 6 | 511 | −5.4% |
| 14-060304-sl1 | 135 | 7 | 127 | −5.9% |
| 14-060304-sl1 | 540 | 7 | 483 | −10.6% |
| 14-060304-sl1 | 135 | 8 | 138 | 2.2% |
| 14-060304-sl1 | 540 | 8 | 456 | −15.6% |
| 14-060304-sl1 | 135 | 9 | 138 | 2.2% |
| 14-060304-sl1 | 540 | 9 | 524 | −3.0% |
| 14-060304-sl1 | 135 | 10 | 136 | 0.7% |
| 14-060304-sl1 | 540 | 10 | 515 | −4.6% |

NA. Not applicable

Further, the results of the recovery of MYO-029 in the presence of GDF-8 were run in duplicate and quantitated. No effect on the ability of the ELISA to detect MYO-029 was observed when any of the MYO-029 samples were co-incubated with GDF-8 at 0, 1, 2, or 10 ng/mL. The observed concentrations were within 20% of the expected values. When MYO-029 samples were co-incubated with GDF-8 at 1000 ng/mL, the observed concentrations of MYO-029 were ≦40% (bias) of the expected concentration. However since GDF-8 may be present at <1 ng/mL, the data suggests that circulating GDF-8 should not compromise the sensitivity of the assay. In an experiment in which 2 mg/kg of MYO-029 was administered subcutaneously to rats, MYO-029 was detected and quantitated as follows:

TABLE 3
Detection of MYO-029 After Administration

| | | | | Calculated | | |
|---|---|---|---|---|---|---|
| | Absorb- | | | Nominal | | Serum |
| | ance | Mean | | MYO-029 | Dilution | Conc. |
| Sample | (A450) | Absorbance | CV | ng/ml | Factor | ng/mL |
| Rat #44, | 2.138 | 2.00445 | 9.42 | 62.2 | 300 | 18669.6 |
| week 1 | 1.8709 | | | | | |
| Rat #45, | 0.9593 | 0.95055 | 1.3 | 26.7 | 600 | 16038.9 |
| week 4 | 0.9418 | | | | | |

In this experiment the following curve parameters were obtained: Min.=0.117195; Max.=3.73079; Slope=1.53126; Ed50=58.7133; and R-Squared=9988.

### Example 3

GDF-8 was biotinylated as follows. Full length GDF-8 was expressed in a fed-batch CHO cell culture bioreactor process, providing the latent complex form of GDF-8. The cell culture harvest was clarified using normal flow microporous filtration and then concentrated and diafiltered using tangential flow ultrafiltration. This retentate pool was then loaded onto Ni²⁺-NTA immobilized metal affinity chromatography (IMAC) where the GDF-8 complex is captured. Elution occurred with a 50 mM Na₂HPO₄, 300 mM NaCl, 20-500 mM imidazole linear gradient over 5 column volumes. The resulting peak then underwent buffer-exchange via dialysis to allow IMAC-derived imidazole removal and to put an appropriate buffer in place for the biotinylation reaction.

The latent complex preparation was then biotinylated. A target sulfo-NHS-LC-biotin to GDF-8 complex molar ratio of 14:1 was used in the reaction. Reagent to substrate ratios of 10:1, 15:1, and 20:1 have also been tested, for example. Solid biotin reagent (EZ-Link Sulfo-NHS-Biotin, Pierce Biotechnology) was dissolved in dimethyl sulfoxide (DMSO) at 200 g/L before it was added to the GDF-8 complex sample. The reaction is performed with a GDF-8 complex concentration of less than 1.5 g/L in 100 mM Na2HPO4, 150 mM NaCl, pH 7.2, at 4° C., for 120 minutes. The reaction mixture was mixed gently at the start of the reaction and shielded from light during the course of the reaction. The reaction was stopped by adding 0.5% (v/v) ethanol amine or 5.0% (v/v) 1000 mM Tris.

This biotinylated GDF-8 complex was then buffer-exchanged via dialysis into a low pH, high chaotrope concentration buffer (6000 mM urea, 300 mM NaCl, 50 mM H₃PO₄, pH=2.5). Dissociation of the complex occurs with protonation at low pH. In this buffer, the complex dissociates and solubilizes into propeptides and mature dimers. Also, free biotin is removed during the dialysis. This retentate pool was then loaded onto high performance size exclusion chromatography where the mature dimer form of GDF-8 is separated from propeptides and residual monomer.

This fraction comprising the biotinylated, mature dimer form of GDF-8 was then further processed on butyl high performance reversed phase chromatography using a 0-90% (v/v) CH₃CN, 0.1% (v/v) CF₃CO₂H, pH=2.0 linear gradient over 5 column volumes. The peak from this step was buffer-exchanged via dialysis into a low pH formulation buffer (0.1% (v/v) CF₃CO₂H, pH=2.0).

The biotinylated mature GDF-8 dimer was assessed for retention of function, for example its activity in binding and reporter gene assays. The biotinylated mature GDF-8 protein was also measured by reversed-phase high performance liquid chromatography/electrospray-ionization quadrupole time-of-flight mass spectrometry (RP-HPLC/ESI-QTOF-MS), and the preparation contained a mix of molar ratios of approximately 0-3, with the majority of the molecules being at 1:1. Higher target molar ratios have yielded measurements as high as 9:1, by adjustment of conditions well known in the art.

MYO-029 is biotinylated using a similar assay, and may be used in the methods of described herein. Essentially, isolated MYO-029 is diluted, buffer-exchanged, and then biotinylated. The reaction and storage conditions are the same as for GDF-8, except for a few parameters. The MYO-029 concentration value ranges from 10-24 g/L. A target sulfo-NHS-LC-biotin (Pierce) to MYO-029 molar ratio in the biotinylation reaction is 40:1, which yields a measured molar ratio of 8-11. This is measured by an avidin:HABA A₆₀₀ nm spectrophotometry assay (Immunopure Avidin and HABA, Pierce). Using dialysis, this reagent is then buffer-exchanged into a low salt, neutral pH formulation buffer (137 mM NaCl, 1 mM KCl, 8 mM Na₂HPO₄, 3 mM KH₂PO₄, pH=7.2).

### Example 4

In one embodiment of the methods provided herein, a GDF-8 modulating agent is detected with a competitive binding ELISA. In this assay, agents that block the binding of GDF-8 to ActRIIB (or another GDF-8 binding partner, such as a GDF-8 receptor) are identified and quantified. This assay includes the steps of contacting a GDF-8 binding partner as a capture agent to a surface, adding GDF-8 in the presence and absence of a biological sample, and detecting complex formation.

In a particular embodiment, GDF-8 latent complex is biotinylated at a ratio of 20 moles of EZ-link Sulfo-NHS-Biotin (Pierce) to 1 mole of the GDF-8 for 2 hours on ice. The reaction is terminated by dropping the pH using 0.5% TFA and the complex is subjected to chromatography on a C₄ Jupiter 250×4.6 mm column (Phenomenex) to separate mature GDF-8 from GDF-8 propeptide. Biotinylated mature GDF-8 fractions eluted with a TFA/CH₃CN gradient are pooled, concentrated and quantified by MicroBCA protein Assay Reagent Kit (Pierce).

Recombinant ActRIIB-Fc chimera (R&D Systems) is coated on 96-well flat-bottom assay plates (Costar) at 1 μg/mL in 0.2 M sodium carbonate buffer overnight at 4° C. Plates are then blocked with 1 mg/mL bovine serum albumin and washed following standard ELISA protocol. 100 μl aliquots of biotinylated GDF-8 at various concentrations (such as 10 ng/mL) with or without a GDF-8 inhibitor (such as at concentrations ranging from 10⁻¹¹M to 10⁻⁷ M) may be added to the blocked ELISA plate, incubated for 1 hr, washed, and the amount of bound GDF-8 detected by streptavidin-horseradish peroxidase (SA-HRP, BD PharMingen) followed by the addition of TMB (KPL, Gaithersburg, Md., Cat. No. 50-76-04). Colorimetric measurements may be done at 450 nm in a microplate reader.

### Example 5

### Reporter Gene Assay

A GDF-8 modulating agent is detected in cell based reporter gene assay (RGA) for biological activity of GDF-8.

The human rhabdomyosarcoma cell line A204 was used, in which A204 (ATCC HTB-82) was stably transfected with a reporter gene construct, pGL3(CAGA)₁₂ (described in U.S. Patent Publ. Nos. 2003/0138422 A1 and 2004/0142382 A1) using well known techniques. Alternatively, A204 cells are transiently transfected with pGL3(CAGA)₁₂ using FuGENE™ 6 transfection reagent (Boehringer Manheim, Germany). Following transfection, cells were cultured on 96 well plates in McCoy's 5A medium supplemented with 2 mM glutamine, 100 U/mL streptomycin, 100 μg/mL penicillin and 10% fetal calf serum for 16 hrs. Cells were treated with or without a constant amount of (75 ng/mL) mature GDF-8 protein and a dilution series of positive control in McCoy's 5A media with glutamine, streptomycin, penicillin, and 10% fetal calf serum for 6 hrs at 37° C. for controls. Optionally, an amount of GDF-8 is selected that provides approximately 80% of the maximal luciferase signal. MYO-029 was preincubated with the GDF-8 for 1 hour at room temperature, and then the proteins are added in the RGA. MYO-029 was assayed at concentrations ranging from 0.1 pM to 10 nM to generate a positive control titration of the GDF-8 modulating agent. Luciferase was quantified in the treated cells using the Luciferase Assay System (Promega). In this assay, 75 ng/mL GDF-8 provides 80% activation while 400 ng/mL of MYO-029 provides 80% inhibition of the reporter gene construct.

In parallel reactions, cells are treated with and without 75 ng/mL of mature GDF-8 protein and with and without test biological samples. Human serum is obtained from individuals undergoing MYO-029 treatment, and diluted 1:5, 1:10, 1:15, 1:20, and 1:40 in buffer. For dilutions lower than 1:10, the test sample serum is further diluted in buffer containing 10% human serum (Bioreclamation, Inc.)

### Example 6

### Antibodies to MYO-029

Neutralizing antibodies to MYO-029, including antibodies to the antigen binding site of MYO-029, were developed as follows: Rabbits were immunized with either intact MYO-029 or MYO-029 protein fragments comprising the MYO-029 binding site. Protease digestion was performed to remove the Fc portion of the MYO-029 antibody in order to avoid generation of a strong immune response in the rabbit to the constant region of this human antibody. Two rabbits were immunized with either the intact or the digested MYO-029. Bleeds were tested for neutralizing activity using ligand binding assays. This procedure produced neutralizing antibodies. All four animals developed good antibody titer results and a positive control rabbit serum was produced by pooling bleeds from all four animals.

All publications, patents, and biological sequences cited in this disclosure are incorporated by reference in their entirety. To the extent the material incorporated by reference contradicts or is inconsistent with the present specification, the present specification will supersede any such material. The citation of any references herein is not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

The embodiments within the specification provide an illustration of embodiments of the invention and should not be construed to limit the scope of the invention. The skilled artisan readily recognizes that many other embodiments are encompassed by the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method to detect an exogenous GDF-8 modulating agent in a biological sample, the method comprising:
(a) adding the biological sample from an individual to be tested to an in vitro assay for a GDF-8 activity;
(b) detecting modulation of the GDF-8 activity; and
(c) comparing the modulation of the GDF-8 activity in the presence of the biological sample to the modulation of the GDF-8 activity in the presence of a control biological sample;
thereby detecting the presence of the exogenous GDF-8 modulating agent in the biological sample.

2. The method of claim 1, further comprising quantitating the level of the GDF-8 modulating agent in the biological sample by comparing the modulation of GDF-8 activity by the biological sample from an individual to a plurality of control samples, each comprising a known concentration of the GDF-8 modulating agent.

3. The method of claim 1, wherein the biological sample comprises a sample from an individual to whom a GDF-8 modulating agent has been or is suspected of having been administered.

4. The method of claim 3, wherein the individual is a mammal, bird, reptile, or fish.

5. The method of claim 4, wherein the individual is a mammal.

6. The method of claim 5, wherein the mammal is a human.

7. The method of claim 4, wherein the biological sample is chosen from serum, blood, plasma, biopsy sample, tissue sample, cell suspension, saliva, oral fluid, cerebrospinal fluid, amniotic fluid, milk, colostrum, mammary gland secretion, lymph, urine, sweat, lacrimal fluid, gastric fluid, synovial fluid, and mucus.

8. The method of claim 7, wherein the biological sample is chosen from serum, blood, and plasma.

9. The method of claim 1, wherein the GDF-8 modulating agent is an antibody that specifically binds to a GDF-8 protein.

10. The method of claim 9, wherein the GDF-8 modulating agent is MYO-029.

11. The method of claim 1, wherein the in vitro assay is an immunoassay.

12. The method of claim 11, wherein the immunoassay comprises:
(a) contacting a GDF-8 protein with a surface of a reaction vessel, wherein the GDF-8 protein is a mature GDF-8 protein dimer;
(b) adding the biological sample to the reaction vessel;
(c) adding a detection agent; and
(d) detecting a GDF-8 modulating agent/GDF-8 protein complex associated with the surface of the reaction vessel.

13. The method of claim 12, wherein the GDF-8 protein comprises a biotin moiety and contacts the surface via the biotin moiety.

14. The method of claim 13, wherein the molar ratio of biotin moiety to GDF-8 protein is less than about 5:1, and wherein the mature GDF-8 dimer is biotinylated as part of a latent GDF-8 complex.

15. The method of claim 14, wherein the molar ratio of biotin moiety to GDF-8 protein is between about 0.5:1 and about 4:1.

16. The method of claim 13, wherein avidin or streptavidin is adsorbed to the surface of the reaction vessel prior to addition of the GDF-8 protein.

17. The method of claim 11, wherein the immunoassay comprises:
(a) contacting a soluble GDF-8 receptor with a surface of a reaction vessel;
(b) adding the biological sample to the reaction vessel;
(c) adding a labeled GDF-8 protein to the reaction vessel; and
(d) detecting the amount of labeled GDF-8 protein/GDF-8 receptor complex associated with the surface in the presence and absence of the biological sample,
wherein a reduction in the amount of labeled GDF-8 protein/GDF-8 receptor complex in the presence of the biological sample detects an exogenous GDF-8 modulating agent in the biological sample.

18. The method of claim 17, further comprising the step of incubating the biological sample with the labeled GDF-8 protein prior to adding the sample to the reaction vessel.

19. The method of claim 18, wherein the labeled GDF-8 protein comprises a biotin moiety.

20. The method of claim 19, wherein the molar ratio of biotin moiety to GDF-8 protein is less than about 5:1.

21. The method of claim 20, wherein the molar ratio of biotin moiety to GDF-8 protein is between about 0.5:1 and about 4:1.

22. The method of claim 1, wherein the in vitro assay is a cell-based reporter gene assay.

23. The method of claim 22, further comprising:
(a) providing a host cell comprising a reporter gene construct in a reaction vessel, wherein the construct comprises a GDF-8-responsive control element and a reporter gene;
(b) adding the biological sample to the reaction vessel; and
(c) detecting reporter gene expression in the cell in the presence and absence of the biological sample,
thereby detecting an exogenous GDF-8 modulating agent.

24. The method of claim 23, further comprising adding a substrate that changes color, luminescence, or fluorescence in the presence of the reporter gene.

25. The method of claim 1, wherein the GDF-8 modulating agent is chosen from:
(a) an antibody that specifically binds to GDF-8;
(b) an antibody that specifically binds to a GDF-8 binding partner;
(c) a GDF-8 receptor;
(d) an ActRIIB protein;
(e) a follistatin-domain containing protein;
(f) a follistatin protein;
(g) a GASP-1 protein;
(h) a GDF-8 protein;
(i) a GDF-8 propeptide;
(j) a non-proteinacious inhibitor; and
(k) a small molecule.

26. The method of claim 25, wherein the GDF-8 modulating agent is an antibody that specifically binds to GDF-8.

27. The method of claim 26, wherein the GDF-8 modulating agent is MYO-029.

28. A method to detect an exogenous GDF-8 modulating agent in a biological sample, the method comprising:
(a) contacting a mature GDF-8 protein with a surface of a reaction vessel;
(b) adding a biological sample to the reaction vessel;
(c) adding a detection agent to the reaction vessel; and
(d) detecting an GDF-8 modulating agent/GDF-8 protein complex associated with the surface of the reaction vessel,
thereby detecting the exogenous GDF-8 modulating agent in the biological sample.

29. The method of claim 28, wherein the mature GDF-8 protein comprises a biotin moiety and contacts the surface via the biotin moiety.

30. The method of claim 29, wherein the molar ratio of biotin moiety to GDF-8 protein is less than about 5:1.

31. The method of claim 30, wherein the molar ratio of biotin moiety to mature GDF-8 protein is between about 0.5:1 and about 4:1.

32. The method of claim 29, wherein avidin or streptavidin is adsorbed to the surface of the reaction vessel prior to addition of the GDF-8 protein.

33. The method of claim 28, wherein the GDF-8 modulating agent is an antibody that specifically binds to GDF-8.

34. The method of claim 33, wherein the antibody is a monoclonal antibody.

35. The method of claim 34, wherein the antibody is MYO-029.

36. The method of claim 28, wherein the detection agent is chosen from an antibody that specifically binds to the GDF-8 modulating agent and a labeled GDF-8 protein.

37. The method of claim 36, wherein the detection agent is an antibody that specifically binds to the constant region of an immunoglobin.

38. The method of claim 37, wherein the immunoglobulin is a human immunoglobulin.

39. The method of claim 28, further comprising quantitating the level of the GDF-8 modulating agent in the biological sample by comparing the modulation of GDF-8 activity by the test biological sample to a plurality of control samples, each comprising a known concentration of the GDF-8 modulating agent.

40. The method of claim 28, further comprising identifying the exogenous GDF-8 modulating agent.

41. The method of claim 28, wherein the biological sample comprises a sample from an individual to whom a GDF-8 modulating agent has been or is suspected of having been administered.

42. The method of claim 28, wherein the biological sample is from a mammal, bird, reptile, or fish.

43. The method of claim 42, wherein the biological sample is from a mammal.

44. The method of claim 43, wherein the mammal is a human.

45. The method of claim 28, wherein the biological sample is chosen from serum, blood, plasma, biopsy sample, tissue sample, cell suspension, saliva, oral fluid, cerebrospinal fluid, amniotic fluid, milk, colostrum, mammary gland secretion, lymph, urine, sweat, lacrimal fluid, gastric fluid, synovial fluid, and mucus.

46. The method of claim 45, wherein the biological sample is chosen from serum, blood, and plasma.

47. A method to detect an exogenous GDF-8 modulating agent in a biological sample, the method comprising:
(a) contacting a capture agent with a surface of a reaction vessel, wherein the capture agent is chosen from a GDF-8 protein and a protein that specifically binds to a GDF-8 protein;
(b) adding the biological sample to the reaction vessel;
(c) adding a detection agent to the reaction vessel; and
(d) detecting a GDF-8 modulating agent/capture agent complex associated with the surface of the reaction vessel,
thereby detecting an exogenous GDF-8 modulating agent in the biological sample.

48. The method of claim 47, wherein the capture agent is a mature GDF-8 protein comprising a biotin moiety.

49. The method of claim 48, wherein the molar ratio of biotin moiety to GDF-8 protein is less than about 5:1.

50. The method of claim 48, wherein the molar ratio of biotin moiety to mature GDF-8 protein is between about 0.5:1 and about 4:1.

51. The method of claim 47, wherein the capture agent is a protein that specifically binds to a GDF-8 protein chosen from:
(a) an antibody that specifically binds to GDF-8;
(b) a soluble GDF-8 receptor;
(c) an ActRIIB protein;
(d) a follistatin-domain containing protein;
(e) a follistatin protein;
(f) a GASP-1 protein; and
(g) a GDF-8 propeptide.

52. A method to detect a GDF-8 modulating agent in a biological sample, the method comprising:
(a) contacting a GDF-8 receptor with a surface of at least a first and a second reaction vessel;
(b) adding the biological sample and a GDF-8 protein to the first reaction vessel of (a);
(c) adding a control sample and a GDF-8 protein to the second reaction vessel of (a);
(d) adding a detectable marker to the first and second reaction vessels; and
(e) comparing the detectable marker signal in the first reaction vessel to the second reaction vessel.
thereby detecting the GDF-8 modulating agent in the biological sample.

53. A method to detect a GDF-8 modulating agent in a human biological sample, the method comprising:
(a) identifying a human candidate for administration of a GDF-8 modulating agent;
(b) providing a biological sample from the candidate;
(c) adding the biological sample to an in vitro assay for a GDF-8 activity;
(d) detecting modulation of the GDF-8 activity; and
(e) comparing the modulation of the GDF-8 activity in the presence of the test biological sample from the candidate to the modulation of the GDF-8 activity in the presence of a control biological sample.
thereby detecting an exogenous GDF-8 modulating agent.

54. A method to detect MYO-029 in a biological sample, comprising:
(a) contacting a biotinylated mature GDF-8 protein dimer with a surface of a reaction vessel, wherein the GDF-8 protein comprises a mean ratio of biotin to GDF-8 dimer of less than 5:1;
(b) adding the biological sample to the reaction vessel;
(c) adding a labeled antibody that specifically binds to a human immunoglobulin to the reaction vessel; and
(d) detecting a MYO-029/biotinylated GDF-8 protein complex associated with the surface of the reaction vessel,
thereby detecting a MYO-029 in the biological sample.

55. The method of claim 54, wherein the label is chosen from an enzyme, an epitope tag, a radiolabel, biotin, a dye, a fluorescent tag label, and a luminescent label.

56. The method of claim 54, wherein the ratio of biotin to GDF-8 dimer is about 0.5:1 to 4:1.

57. The method of claim 54, wherein the biological sample comprises a sample from an individual to whom a GDF-8 modulating agent has been or is suspected of having been administered.

58. The method of claim 57, wherein the individual is a mammal, bird, reptile, or fish.

59. The method of claim 58, wherein the individual is a mammal.

60. The method of claim 59, wherein the mammal is human.

61. The method of claim 54, wherein the biological sample is chosen from serum, blood, plasma, biopsy sample, tissue sample, cell suspension, saliva, oral fluid, cerebrospinal fluid, amniotic fluid, milk, colostrum, mammary gland secretion, lymph, urine, sweat, lacrimal fluid, gastric fluid, synovial fluid, and mucus.

62. The method of claim 61, wherein the biological sample is chosen from serum, blood, and plasma.

63. The method of claim 54, wherein the labeled antibody specifically binds to the constant region of a human immunoglobulin.
